SEQUENCE LISTING (23 sequences)

SEQ ID NO: 1 - protein, 162 aa, Lolium perenne
MAQRDHSKEEPTEVRAPEITLCANRCGFPGNPATQNLCQNCFLAVARPSSPSSSMPAAVSVSTPAAVVDRPRPAQVELSPSSPAVDQAPALEVKATKTSVNRCSSCRKRVGLTGFRCRCDRTFCGEHRYSDRHGCTFDYRAAARDAIARDNPVVRASKIVRF

SEQ ID NO: 2 - protein, 164 aa, Oryza sativa Indica
MAQRDKKDQEPTELRAPEITLCANSCGFPGNPATQNLCQNCFLAATASTSSPSSLSSPVLDKQPPRPAAPLVEPQAPLPPPVEEMASALATAPAPVAKTSAVNRCSRCRKRVGLTGFRCRCGHLFCGEHRYSDRHGCSYDYKSAARDAIARDNPVVRAAKIVRF

SEQ ID NO: 3 - protein, 164 aa, Oryza sativa Indica
MAQGDKKDQEPTELRAPEITLCANSCGFPGNPATQNLCQNCFLAATASTSSPSSLSSPVLDKQPPRPAAPLVEPQAPLPPPVEEMASALATAPAPVAKTSAVNRCSRCRKRVGLTGFRCRCGHLFCGEHRYSDRHGCSYDYKSAARDAIARDNPVVRAAKIVRF

SEQ ID NO: 4 - protein, 164 aa, Oryza sativa
MAQRDKKDQEPTELRAPEITLCANSCGFPGNPATQNLCQNCFLAATAFTSSPSSLSSPVLDKQPPRPAAPLVEPQAPLPPPVEEMASALATAPAPVAKTSAVNRCSRCRKRVGLTGFRCRCGHLFCGEHRYSDRHGCSYDYKSAARDAIARDNPVVRAAKIVRF

SEQ ID NO: 5 - protein, 170 aa, Oryza sativa
MAQREKKVEEPTELRAPEMTLCANSCGFPGNPATNNLCQNCFLAASASSSSSSAAASPSTTSLPVFPVVEKPRQAVQSSAAAAVALVVERPTAGPVESSSKASRSSSVNRCHSCRRRVGLTGFRCRCGELYCGAHRYSDRHDCSFDYKSAARDAIARENPVVRAAKIVRF

SEQ ID NO: 6 - protein, 160 aa, Zea mays
MAQRDKKEEPTELRAPEITLCANNCGFPGNPATQNLCQSCFSASRSPSSPTSSSSSLASAASQPRPAALVVDAAAVDALAAPAAAAGQATEAAARASASRCSSCWKRVGLTGFRCRCGELFCGAHRYSDRHGCSYDYKGAARDAIAREDPVVRAAKVVRF

SEQ ID NO: 7 - DNA, 639 bp, Lolium perenne
aagccagccagactctctctcgtaccgcgacgtacgcgcggactctccggaccagacagagcacagcctgcccgttcgttccttcccatggcgcagcgcgaccacagcaaggaggagccgacggaggtgcgggcgccggagatcacgctctgcgccaaccgctgcggcttcccgggcaacccggccacgcagaacctctgccagaactgcttcctcgccgtcgcacgcccgtcctcgccgtcctcctccatgcctgcggcagtctccgtctccacccccgcggcggtcgtcgacaggccgaggccggcgcaggttgagctatcgccgtccagccccgccgttgaccaagctccggcgctggaggtgaaggcgacaaagacgtcggtgaaccggtgctccagctgccggaagcgcgtcgggctgaccgggttccggtgccgctgcgaccggacgttctgcggcgagcaccgctactcggaccgccacggctgcaccttcgactacagggcggccgccagggacgccatcgccagggacaaccccgtggtgcgcgcgtccaagatcgtcaggttctgagagagaatcgacggactttagcccaaggcaaagagaagaatgaacggaggaaactgaaggtgc

SEQ ID NO: 8 - DNA, 844 bp, Oryza sativa Indica
gatctctcctgcaatcctcatcacacagcaaacccaaaccgcgagcggaatcctcagcctgctgagagagcctgagaccaagagggggattcttttttggttattgacgatggcgcagcgcgacaagaaggatcaggagccgacggagctcagggcgccggagatcacgctgtgcgccaacagctgcggattcccgggcaacccggccacgcagaacctctgccagaactgcttcttggcggccacggcgtccacctcgtcgccgtcttctttgtcgtcaccggtgctcgacaagcagccgccgaggccggcggcgccgctggttgagcctcaggctcctctcccaccgcctgtggaggagatggcctccgcgctcgcgacggcgccggcgccggtcgccaagacgtcggcggtgaaccggtgctccaggtgccggaagcgtgtcggcctcaccgggttccggtgccggtgcggccacctgttctgcggcgagcaccggtactccgaccgccacggctgcagctacgactacaagtcggcggcaagggacgccatcgccagggacaacccggtggtgcgcgcggccaagatcgttaggttctgagaggcaaacaaaattaaaaaaaaaatctactgttttagcaagaaatggagaaaaaaattgggaattgaaggtgtggatgttattattatgctgttctcttctcgcaattgtttttccctttttattctttttaattgcaaacgggaggataagtggtggaaaaggaatagtgtaacaataatggtgatgtgaggtggttgagggaaaaagaatcgaagaacaaaaaaaaaaaaaaaaaaa

SEQ ID NO: 9 - DNA, 495 bp, Oryza sativa Indica
atggcgcagggcgacaagaaggatcaggagccgacggagctcagggcgccggagatcacgctgtgcgccaacagctgcggattcccgggcaacccggccacgcagaacctctgccagaactgcttcttggcggccacggcgtccacctcgtcgccgtcttctttgtcgtcaccggtgctcgacaagcagccgccgaggccggcggcgccgctggttgagcctcaggctcctctcccaccgcctgtggaggagatggcctccgcgctcgcgacggcgccggcgccggtcgccaagacgtcggcggtgaaccggtgctccaggtgccggaagcgtgtcggcctcaccgggttccggtgccggtgcggccacctgttctgcggcgagcaccggtactccgaccgccacggctgcagctacgactacaagtcggcggcgagggacgccatcgccagggacaacccggtggtgcgcgcggccaagatcgttaggttctga

SEQ ID NO: 10 - DNA, 495 bp, Oryza sativa
atggcgcagcgcgacaagaaggatcaggagccgacggagctcagggcgccggagatcacgctgtgcgccaacagctgcggattcccgggcaacccggccacgcagaacctctgccagaactgcttcttggcggccacggcgttcacctcgtcgccgtcttctttgtcgtcaccggtgctcgacaagcagccgccgaggccggcggcgccgctggttgagcctcaggctcctctcccaccgcctgtggaggagatggcctccgcgctcgcgacggcgccggcgccggtcgccaagacgtcggcggtgaaccggtgctccaggtgccggaagcgtgtcggcctcaccgggttccggtgccggtgcggccacctgttctgcggcgagcaccggtactccgaccgccacggctgcagctacgactacaagtcggcggcgagggacgccatcgccagggacaacccggtggtgcgcgcggccaagatcgttaggttctga

SEQ ID NO: 11 - DNA, 933 bp, Oryza sativa
ctaacccattcccaaaagcaaagcgaacctaagctcgcttcgtctctctctcggattcgatcatggcgcagagggagaagaaggtggaggagccgacggagctgagggcgccggagatgacgctctgcgccaacagctgcgggttcccgggcaacccggcgaccaacaacctctgccagaactgcttcttggctgcctcggcgtcttcttcttcttcttccgccgctgcctcgccgtcgacgacgtcgttgccggtgtttccggtggtggagaagccgaggcaggccgtacagtcgtcggcggcggcggcggtggcgctggtggttgagcggccgacggcggggccggtggagtcgtcgtcgaaggcgtcgaggtcgtcgtcggtcaaccgatgccacagctgccggaggcgggtgggcctgaccgggttccggtgccgctgcggcgagctctactgcggcgcgcaccggtactccgaccgccacgactgcagcttcgactacaagtcggcggcgagggacgccatcgccagggagaaccccgtcgtccgcgccgccaagatcgttaggttctaaaaggataatacaaggggaaaccaacctgaattttcctcctttttctttcttttttcttcttctttccttttaaaaattaaagttgagggtattattttttcttttcctgagagcttcttaaaggtgaaaggggaaaaaaaagaaagaaaaagaaaaggagatgagatgggtgactcaagatgcatcaagcaaaggaagaacaatatgatgatgatgatgttgttgttgtagtggtggttgaggttggaagttggatggaaagagggaggaaggaagaagatggggttgttgtgttgtcctcccacaaatctctatttaattttaattcccttttgtataaaatttattattatctgtg

SEQ ID NO: 12 - DNA, 483 bp, Zea mays
atggcgcagcgcgacaagaaggaggagcccacggagctccgggcgccggagatcacgctctgcgccaacaactgcgggttcccgggtaacccggccacgcagaacctgtgccagagctgcttctcggcgtcgaggtcgccgtcgtcccctacctcctcctcctcttccctggcgtccgcggcgtcgcagccgaggccagcagcgctcgtcgtagacgcggcagcggtggatgcgctggccgctccggcggccgcggccggccaggctacggaagccgcggcgagggcgtccgcgagccggtgctccagctgctggaagcgcgtggggctgacggggttccggtgccgctgcggcgagctcttctgcggcgcgcaccggtactcggaccggcacggctgcagctacgactacaagggcgccgcccgggacgccatcgcccgggaggacccggtggtgcgcgcggccaaggtcgttaggttctga

SEQ ID NO: 13 - DNA, 10 bp, Lolium perenne
cctgcggcag

SEQ ID NO: 14 - DNA, 26 bp, Lolium perenne
aagccagccagactctctctcgtacc

SEQ ID NO: 15 - DNA, 26 bp, Lolium perenne
gcaccttcagtttcctccgttcattc

SEQ ID NO: 16 - protein, 25 aa, Lolium perenne
ITLCANRCGFPGNPATQNLCQNCFL

SEQ ID NO: 17 - protein, 9 aa, Artificial
  motif
CGFPGNPAT

SEQ ID NO: 18 - protein, 33 aa, Lolium perenne
CSSCRKRVGLTGFRCRCDRTFCGEHRYSDRHGC

SEQ ID NO: 19 - protein, 11 aa, Artificial
  motif
RVGLTGFRCRC

SEQ ID NO: 20 - DNA, 10623 bp, Artificial
  vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaataagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaaggccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctccattagaagccagccagactctctctcgtaccgcgacgtacgcgcggactctccggaccagacagagcacagcctgcccgttcgttccttcccatggcgcagcgcgaccacagcaaggaggagccgacggaggtgcgggcgccggagatcacgctctgcgccaaccgctgcggcttcccgggcaacccggccacgcagaacctctgccagaactgcttcctcgccgtcgcacgcccgtcctcgccgtcctcctccatgcctgcggcagtctccgtctccacccccgcggcggtcgtcgacaggccgaggccggcgcaggttgagctatcgccgtccagccccgccgttgaccaagctccggcgctggaggtgaaggcgacaaagacgtcggtgaaccggtgctccagctgccggaagcgcgtcgggctgaccgggttccggtgccgctgcgaccggacgttctgcggcgagcaccgctactcggaccgccacggctgcaccttcgactacagggcggccgccagggacgccatcgccagggacaaccccgtggtgcgcgcgtccaagatcgtcaggttctgagagagaatcgacggactttagcccaaggcaaagagaagaatgaacggaggaaactgaaggtgcggcctggtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtatcggcgcgccttaattaaggcgcgccctgca

SEQ ID NO: 21 - DNA, 11002 bp, Artificial
  vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaaggcgcgccgatactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaccaggccgcaccttcagtttcctccgttcattcttctctttgccttgggctaaagtccgtcgattctctctcagaacctgacgatcttggacgcgcgcaccacggggttgtccctggcgatggcgtccctggcggccgccctgtagtcgaaggtgcagccgtggcggtccgagtagcggtgctcgccgcagaacgtccggtcgcagcggcaccggaacccggtcagcccgacgcgcttccggcagctggagcaccggttcaccgacgtctttgtcgccttcacctccagcgccggagcttggtcaacggcggggctggacggcgatagctcaacctgcgccggcctcggcctgtcgacgaccgccgcgggggtggagacggagactgccgcaggcatggaggaggacggcgaggacgggcgtgcgacggcgaggaagcagttctggcagaggttctgcgtggccgggttgcccgggaagccgcagcggttggcgcagagcgtgatctccggcgcccgcacctccgtcggctcctccttgctgtggtcgcgctgcgccatgggaaggaacgaacgggcaggctgtgctctgtctggtccggagagtccgcgcgtacgtcgcggtacgagagagagtctggctggcttctaatggcttgcacaagtgatattgcactgattttacctcacgaaatctagctagtacgtcttgtttagtacactgcgataagtagctgtgtttgttgtgtaagcagtgatggccgaggacatacttataagcagcttgaggagctgggataggcatcgctgttgcatcgatcaagcgccacgatagggcgctacgtgtgagttggtaggcggaaagcctgtgactgctgactcgcccacgacgagaagccgggcgtgtgcagatagtcggcgcgtcggacgaagtcgccacgtctcggctccttctggaagcatggacaggtgaccgatagctcgcatgcatcgattatccaatacaagactgatattcgtccatcgtctgttaaactagcacggtggcgagatgggcaatattctatatatcagttttgataattttaagtatcggaacttttaacaagtattagttgactttgtagccattggatttgtctcttgattcatgttaatcttacacagttactcagaagattttcatccagtacaaaataatttttcattcaatacagaacaattgtttatgggtaaaaatgtttatctactagtacaaaacttattttgttcacatcccattgcccatctcacaaagaattcacgtcccacaaatactaggatgagtgggtgacatcctgacatcagcatgtgcgtccattatattatgccgtctatcattacgggaaaaaaagctcaatgccgtgccgggctctttgccgtgtgctgtcagcgccctttgccatgcgaaacttctttcccgtgtgcctcgtgcacggcaatgaattctttgccgtacagctctgggaggacgcacggcaaacaatacgcgcacggcaaagcgccagctcagcgctcggcaaaggcagcatcccacggcaaagaaataaaaggcacacggcaaagaacgcttcacggcaaagatagagccaaggcgcacggcaaagacgtggacacggcaacgatgcctttgccgtgcgggccaacccattgccgtggacacggcaacgatgcctttgccgtgcgggccaacccattgccgtgcatttttgcacggcacagagggaaattttatttattttccttctattgtattatatttatattaatacttatatttgttttaccagcccgttaattaaggcgcgccctgca

SEQ ID NO: 22 - DNA, 21 bp, Artificial
  primer
aatacgaggtcgccaacatct

SEQ ID NO: 23 - DNA, 23 bp, Artificial
  primer
aggaaccctaattcccttatctg

(19) United States
(12) Patent Application Publication
(10) Pub. No.: US 2011/0185452 A1
(43) Pub. Date: Jul. 28, 2011

(54) COMPOSITIONS AND METHODS FOR IMPROVING PLANTS

(76) Inventors: Sathish Puthigae, Auckland (NZ); Catherine Jane Bryant, Auckland (NZ); Shivendra Bajaj, Auckland (NZ); Kerry Robert Templeton, Auckland (AZ)

(21) Appl. No.: 12/936,194

(22) PCT Filed: May 28, 2009
(86) PCT No.: PCT/NZ2009/000087

§ 371 Date: Apr. 10, 2011

Related U.S. Application Data
(60) Provisional application No. 61/058,486, filed on Jun. 3, 2008.

(51) Int. Cl. C12N 15/82; C07H 21/00; C07K 14/415; C12N 15/63; C12N 5/10; A01H 5/00; C12Q 1/68; C12Q 1/02; A01H 5/08; A01H 5/10

(52) U.S. Cl. 800/289; 536/23.6; 530/372; 435/320.1; 435/419; 800/298; 800/290; 435/6.1; 435/29

(57) ABSTRACT

The invention provides an isolated polynucleotide encoding a polypeptide with the sequence of SEQ ID NO: 1 or a variant thereof, wherein the variant is a polypeptide capable of modulating in a plant at least one of: i) biomass, ii) seed yield, and III) tolerance to at least one environmental stress selected from drought, cold, freezing, heat and salinity. The invention also provides, construct, vectors, host cells, plant cells and plants genetically modified to comprise the polynucleotide. The invention also provides methods for producing and selecting plants that are altered for at least one of: i) biomass, ii) seed yield, and iii) tolerance to at least one environmental stress selected from drought, cold, freezing, heat and salinity, making use of the polynucleotides of the invention.

### TECHNICAL FIELD

The present invention relates to compositions and methods for producing plants with improved biomass and/or seed yield and/or stress tolerance.

### BACKGROUND ART

As the population of the world increases, a major goal of agricultural research is to improve the biomass yield and seed yield of crop and forage plant species.

Such improvements have until recently depended on selective breeding of plants for desirable characteristics. However for many plants the heterogeneous genetic complements produced in off-spring do not result in the same desirable traits as those of their parents, thus limiting the effectiveness of selective breeding approaches.

Advances in molecular biology now make it possible to genetically manipulate the germplasm of both plants and animals. Genetic engineering of plants involves the isolation and manipulation of genetic material and the subsequent introduction of such material into a plant. This technology has led to the development of plants that are capable of expressing pharmaceuticals and other chemicals, plants with increased pest resistance, increased stress tolerance, and plants that express other beneficial traits.

Whilst it is known in the art that certain growth factors may be applied to increase plant size, the application of such growth factors is both costly and time consuming. Thus, there exists a need for plants with increased biomass relative to their cultivated counterparts.

Improvements in the grain yield of plant crop plants may be achieved by developing plants that produce more seed or grain than the equivalent wild-type plants.

Thus, there exists a need for plants with increased seed yield relative to their normally cultivated counterparts.

Environmental abiotic stresses, including drought stress, cold stress, freezing stress, heat stress and salinity stress are major factors limiting plant growth and productivity. Crop losses and reduction in biomass of major crops including maize, wheat and rice caused by such stresses represent significant economic issues and also lead to food shortages in several underdeveloped countries.

The development of stress tolerant plants has the potential to reduce or solve at least some of these problems. The use of traditional plant breeding strategies to produce new lines of plants that exhibit tolerance to these types of stresses has been slow. Lack of sufficient germplasm resources and incompatibility between distantly related plant species, present significant problems in conventional breeding. Further, the cellular processes leading to tolerance to such stresses are complex and involve multiple mechanisms of cellular adaptation and numerous metabolic pathways. This limits the success of both traditional breeding and that of genetic engineering approaches to development of stress tolerant plants. It would be beneficial to identify genes and proteins involved in controlling the complex processes leading to stress tolerance.

Regulators of gene expression, such as transcription factors, involved in controlling stress tolerance may be particularly useful in genetic engineering of plants, as a single gene may control a whole cascade of genes leading to the tolerance phenotype. Furthermore, there is sometimes commonality in many aspects of the different types of stress tolerant responses referred above. For example, genes that increase tolerance to cold or salt may also improve drought stress tolerance. This has been demonstrated in the case of the transcription factor At CBF/DREB 1 (Kasuga et al., 1999 Nature Biotech 17: 287-91) and the vacuolar pyrophosphatase AVP1 (Gaxiola et al., 2001 PNAS 98:11444-19).

Whilst some potentially useful genes have been identified, the identification and cloning of plant genes that confer tolerance to stress remains fragmented and incomplete. Although it is assumed that stress induced proteins may have a role in stress tolerance, evidence is still lacking and the function of many such stress responsive genes is unknown.

It would be beneficial to identify genes which have the capacity to confer stress tolerance in stress susceptible plant species. The development of stress tolerant crops will provide many advantages such as increasing biomass and producing plants that may be cultivated in previously unsuitable environmental conditions. Thus, there exists a need for compositions and methods for producing plants with improved stress tolerance relative to cultivated counterparts.

It is an object of the invention to provide improved compositions and/or methods for developing plant varieties with at least one of
i) altered biomass,
ii) altered seed yield, and
iii) altered tolerance to at least one of the following stresses; drought, cold, freezing, heat and salinity,
   or at least to provide the public with a useful choice.

### SUMMARY OF THE INVENTION

### Polynucleotides Encoding Polypeptides

In one aspect the invention provides an isolated polynucleotide encoding a polypeptide with the sequence of SEQ ID NO: 1 or a variant thereof, wherein the variant is a polypeptide capable of modulating in a plant at least one of:
i) biomass,
ii) seed yield, and
iii) tolerance to at least one environmental stress selected from drought, cold, freezing, heat and salinity.

Preferably the polypeptide is capable of modulating both biomass and seed yield.

Preferably the polypeptide is capable of modulating both biomass and tolerance to at least one of the recited environmental stresses.

Preferably the polypeptide is capable of modulating biomass, seed yield and tolerance to at least one of the recited environmental stresses.

In a further aspect the invention provides an isolated polynucleotide encoding a polypeptide with the sequence of SEQ ID NO: 1 or a variant thereof, wherein the variant is a polypeptide capable of modulating biomass in a plant.

In a further aspect the invention provides an isolated polynucleotide encoding a polypeptide with the sequence of SEQ ID NO: 1 or a variant thereof, wherein the variant is a polypeptide capable of modulating seed yield in a plant.

In a further aspect the invention provides an isolated polynucleotide encoding a polypeptide with the sequence of SEQ ID NO: 1 or a variant thereof, wherein the variant is a polypeptide capable of modulating in a plant tolerance to at least one environmental stress selected from drought, cold, freezing, heat and salinity.

Preferably the polypeptide is capable of modulating at least two, preferably at least three, more preferably at least four, and most preferably all five of the recited environmental stresses.

In one embodiment of each of the three aspects above, the isolated polynucleotide encodes a polypeptide with at least 70% identity to the sequence of SEQ ID NO:1.

In a further embodiment the polypeptide comprises an A20-type zinc finger domain and an AN1-type zinc finger domain.

Preferably the A20-type domain is in the N terminal half of the polypeptide.

Preferably the AN1-type domain is in the C terminal half of the polypeptide.

Preferably the A20-type domain has the general formula: X3-C-X(2-4)-C-X11-C-X2-C-X2, where X can be any amino acid.

Preferably the AN1-type domain has the general formula: C-X2-C-X(9-12)-C-X(1-2)-C-X4-C-X2-H-X5-H-X-C, where X can be any amino acid.

Preferably the A20-type domain has at least 70% identity to sequence of SEQ ID NO:16.

Preferably the A20-type domain comprises the sequence of SEQ ID NO:17.

Preferably the polypeptide comprises the sequence of SEQ ID NO:17.

Preferably the A20-type domain consists of the sequence of SEQ ID NO:16.

Preferably the AN1-type domain has at least 70% identity to sequence of SEQ ID NO:18.

Preferably the AN1-type domain comprises the sequence of SEQ ID NO:19.

Preferably the polypeptide comprises the sequence of SEQ ID NO:19.

Preferably the AN1-type domain consists of the sequence of SEQ ID NO:18.

In a further embodiment the isolated polynucleotide encodes a polypeptide with the sequence of SEQ ID NO:1.

In a further embodiment the isolated polynucleotide comprises a sequence with at least 70% identity to the coding sequence of SEQ ID NO:7.

In a further embodiment the isolated polynucleotide comprises the coding sequence of SEQ ID NO: 7.

In a further embodiment the isolated polynucleotide comprises a sequence capable of hybridising under stringent conditions to the coding sequence of SEQ ID NO:7.

In a further embodiment the isolated polynucleotide comprises the coding sequence of SEQ ID NO: 7.

### Polynucleotides

In a further aspect the invention provides an isolated polynucleotide comprising the sequence of SEQ ID NO: 7 or a variant thereof, wherein the variant encodes a polypeptide capable of modulating in a plant at least one of:
i) biomass,
ii) seed yield, and
iii) tolerance to at least one environmental stress selected from drought, cold, freezing, heat and salinity.

Preferably the peptide is capable of modulating both biomass and seed yield.

Preferably the peptide is capable of modulating both biomass and tolerance to at least one of the recited environmental stresses.

Preferably the peptide is capable of modulating biomass, seed yield and tolerance to at least one of the recited environmental stresses.

In a further aspect the invention provides an isolated polynucleotide comprising the sequence of SEQ ID NO: 7 or a variant thereof, wherein the variant encodes a polypeptide capable of modulating biomass in a plant.

In a further aspect the invention provides an isolated polynucleotide comprising the sequence of SEQ ID NO: 7 or a variant thereof, wherein the variant encodes a polypeptide capable of modulating seed yield in a plant.

In a further aspect the invention provides an isolated polynucleotide comprising the sequence of SEQ ID NO: 7 or a variant thereof, wherein the variant encodes a polypeptide capable of modulating in a plant tolerance to at least one environmental stress selected from drought, cold, freezing, heat and salinity.

Preferably the polypeptide is capable of modulating at least two, generally at least three, more preferably at least four, and most preferably all five of the recited environmental stresses.

In one embodiment of each of the three aspects above, the isolated polynucleotide comprises a sequence with at least 70% identity to the coding sequence of SEQ ID NO:7.

In a further embodiment the polypeptide comprises an A20-type zinc finger domain and an AN1-type zinc finger domain.

Preferably the A20-type domain is in the N terminal half of the polypeptide.

Preferably the AN1-type domain is in the C terminal half of the polypeptide.

Preferably the A20-type domain has the general formula: X3-C-X(2-4)-C-X11-C-X2-C-X2, where X can be any amino acid.

Preferably the AN1-type domain has the general formula: C-X2-C-X(9-12)-C-X(1-2)-C-X4-C-X2-H-X5-H-X-C, where X can be any amino acid.

Preferably the A20-type domain has at least 70% identity to sequence of SEQ ID NO:16.

Preferably the A20-type domain comprises the sequence of SEQ ID NO:17.

Preferably the polypeptide comprises the sequence of SEQ ID NO:17.

Preferably the A20-type domain consists of the sequence of SEQ ID NO:16.

Preferably the AN1-type domain has at least 70% identity to sequence of SEQ ID NO:18.

Preferably the AN1-type domain comprises the sequence of SEQ ID NO:19.

Preferably the polypeptide comprises the sequence of SEQ ID NO:19.

Preferably the AN1-type domain consists of the sequence of SEQ ID NO:18.

In a further embodiment the isolated polynucleotide comprises a sequence capable of hybridising under stringent conditions to the coding sequence of SEQ ID NO:7.

In a further embodiment the isolated polynucleotide comprises the coding sequence of SEQ ID NO: 7.

### Polypeptides

In a further aspect the invention provides an isolated polypeptide with the sequence of SEQ ID NO: 1 or a variant thereof, wherein the variant is a polypeptide capable of modulating in a plant at least one of:
i) biomass,
ii) seed yield, and
iii) tolerance to at least one environmental stress selected from drought, cold, freezing, heat and salinity.

Preferably the peptide is capable of modulating both biomass and seed yield.

Preferably the peptide is capable of modulating both biomass and tolerance to at least one of the recited environmental stresses.

Preferably the peptide is capable of modulating biomass, seed yield and tolerance to at least one of the recited environmental stresses.

In a further aspect the invention provides an isolated polypeptide with the sequence of SEQ ID NO: 1 or a variant thereof, wherein the variant is a polypeptide capable of modulating biomass in a plant.

In a further aspect the invention provides an isolated polypeptide with the sequence of SEQ ID NO: 1 or a variant thereof, wherein the variant is a polypeptide capable of modulating seed yield in a plant.

In a further aspect the invention provides an isolated polypeptide with the sequence of SEQ ID NO: 1 or a variant thereof, wherein the variant is a polypeptide capable of modulating in a plant tolerance to at least one environmental stress selected from drought, cold, freezing, heat and salinity.

Preferably the polypeptide is capable of modulating at least two, generally at least three, more preferably at least four, and most preferably all five of the recited environmental stresses.

In one embodiment of each of the three aspects above, the isolated polypeptide has at least 70% identity to the sequence of SEQ ID NO:1.

In a further embodiment the polypeptide comprises an A20-type zinc finger domain and an AN1-type zinc finger domain.

Preferably the A20-type domain is in the N terminal half of the polypeptide. Preferably the AN1-type domain is in the C terminal half of the polypeptide. Preferably the A20-type domain has the general formula: X3-C-X(2-4)-C-X11-C-X2-C-X2, where X can be any amino acid.

Preferably the AN1-type domain has the general formula: C-X2-C-X(9-12)-C-X(1-2)-C-X4-C-X2-H-X5-H-X-C, where X can be any amino acid.

Preferably the A20-type domain has at least 70% identity to sequence of SEQ ID NO:16.

Preferably the A20-type domain comprises the sequence of SEQ ID NO:17.

Preferably the polypeptide comprises the sequence of SEQ ID NO:17.

Preferably the A20-type domain consists of the sequence of SEQ ID NO:16.

Preferably the AN1-type domain has at least 70% identity to sequence of SEQ ID NO:18.

Preferably the AN1-type domain comprises the sequence of SEQ ID NO:19.

Preferably the polypeptide comprises the sequence of SEQ ID NO:19.

Preferably the AN1-type domain consists of the sequence of SEQ ID NO:18.

In a further embodiment the isolated polypeptide comprises the sequence of SEQ ID NO:1.

In a further aspect the invention provides an isolated polynucleotide comprising a fragment, of at least 50 nucleotides in length, of a polynucleotide of the invention.

In one embodiment the environmental stress is drought.

In a further embodiment the environmental stress is cold.

In a further embodiment the environmental stress is freezing.

In a further embodiment the environmental stress is heat.

In a further embodiment the environmental stress is salinity.

In a further aspect the invention provides a genetic construct comprising a polynucleotide of the invention.

In one embodiment the genetic construct is an expression construct.

In a further aspect the invention provides a vector comprising a polynucleotide, genetic construct or expression construct of the invention.

In a further aspect the invention provides a host cell comprising a polynucleotide, genetic construct or expression construct of the invention.

In a further aspect the invention provides a host cell genetically modified to express a polynucleotide of the invention.

In a further aspect the invention provides a plant cell comprising a genetic construct or the expression construct of the invention.

In a further aspect the invention provides a plant cell genetically modified to express a polynucleotide of the invention.

In a further aspect the invention provides a plant which comprises a plant cell of the invention.

### Methods Using Polynucleotides

In a further aspect the invention provides a method of producing a plant with at least one of:
i) altered biomass,
ii) altered seed yield, and
iii) altered tolerance to at least one environmental stress selected from drought, cold, freezing, heat and salinity,
   the method comprising transformation of a plant cell or plant with:
   a) a polynucleotide comprising the nucleotide sequence of SEQ ID NO:7, or a variant thereof wherein the variant encodes a polypeptide capable of altering biomass, and/or tolerance to at least one of the recited environmental stresses in a plant;
   b) a polynucleotide comprising a fragment, of at least 15 nucleotides in length, of the polynucleotide of a); or
   c) a polynucleotide comprising a complement of the polynucleotide of a) or b).

In a further aspect the invention provides a method of producing a plant with altered biomass,

the method comprising transformation of a plant cell or plant with:
a) a polynucleotide comprising the nucleotide sequence of SEQ ID NO:7, or a variant thereof, wherein the variant encodes a polypeptide capable of altering biomass in a plant;
b) a polynucleotide comprising a fragment, of at least 15 nucleotides in length, of the polynucleotide of a); or
c) a polynucleotide comprising a complement of the polynucleotide of a) or b).

In a further aspect the invention provides a method of producing a plant with altered seed yield,

the method comprising transformation of a plant cell or plant with:
a) a polynucleotide comprising the nucleotide sequence of SEQ ID NO:7, or a variant thereof, wherein the variant encodes a polypeptide capable of altering seed yield in a plant;
b) a polynucleotide comprising a fragment, of at least 15 nucleotides in length, of the polynucleotide of a); or
c) a polynucleotide comprising a complement of the polynucleotide of a) or b).

In a further aspect the invention provides a method of producing a plant with altered tolerance to at least one environmental stress selected from drought, cold, freezing heat and salinity, the method comprising transformation of a plant cell or plant with:
a) a polynucleotide comprising the nucleotide sequence of SEQ ID NO:7, or a variant thereof, wherein the variant encodes a polypeptide capable of increasing tolerance to at least one of the recited environmental stresses in a plant;
b) a polynucleotide comprising a fragment, of at least 15 nucleotides in length, of the polynucleotide of a); or
c) a polynucleotide comprising a complement of the polynucleotide of a) or b).

Altered may be either increased or decreased.

Preferably altered is increased.

Preferably the polypeptide is capable of modulating at least two, generally at least three, more preferably at least four, and most preferably all five of the recited environmental stresses.

In one embodiment of each of the three aspects, the isolated polynucleotide encodes a polypeptide with at least 70% identity to the sequence of SEQ ID NO:1.

In a further embodiment the polypeptide comprises an A20-type zinc finger domain and an AN1-type zinc finger domain.

Preferably the A20-type domain is in the N terminal half of the polypeptide.

Preferably the AN1-type domain is in the C terminal half of the polypeptide.

Preferably the A20-type domain has the general formula: X3-C-X(2-4)-C-X11-C-X2-C-X2, where X can be any amino acid.

Preferably the AN1-type domain has the general formula: C-X2-C-X(9-12)-C-X(1-2)-C-X4-C-X2-H-X5-H-X-C, where X can be any amino acid.

Preferably the A20-type domain has at least 70% identity to sequence of SEQ ID NO:16.

Preferably the A20-type domain comprises the sequence of SEQ ID NO:17.

Preferably the polypeptide comprises the sequence of SEQ ID NO:17.

Preferably the A20-type domain consists of the sequence of SEQ ID NO:16.

Preferably the AN1-type domain has at least 70% identity to sequence of SEQ ID NO:18.

Preferably the AN1-type domain comprises the sequence of SEQ ID NO:19.

Preferably the polypeptide comprises the sequence of SEQ ID NO:19.

Preferably the AN1-type domain consists of the sequence of SEQ ID NO:18.

In a further embodiment the isolated polynucleotide encodes a polypeptide with the sequence of SEQ ID NO:1

Preferably the plant is transformed with a genetic construct or vector comprising the polynucleotide.

In one embodiment the environmental stress is drought.

In a further embodiment the environmental stress is cold.

In a further embodiment the environmental stress is freezing.

In a further embodiment the environmental stress is heat.

In a further embodiment the environmental stress is salinity.

In a further embodiment the variant comprises the sequence of any one of SEQ ID NO:8 to 12.

In a further embodiment the polynucleotide of a) comprises the sequence of SEQ ID NO:1.

### Mthods—Using Polynucleotides Encoding Polypeptides

In a further aspect the invention provides a method of producing a plant with at least one of:
i) altered biomass,
ii) altered seed yield, and
iii) altered tolerance to at least one environmental stress selected from drought, cold, freezing, heat and salinity,
   the method comprising transformation of a plant with:
   a) a polynucleotide encoding of a polypeptide with the amino acid sequence of SEQ ID NO:1 or a variant of the polypeptide, wherein the variant is capable of altering biomass and/or tolerance to at least one environmental stress selected from drought, cold, freezing, heat and salinity in a plant;
   b) a polynucleotide comprising a fragment, of at least 15 nucleotides in length, of the polynucleotide of a); or
   c) a polynucleotide comprising a complement of the polynucleotide of a) or b).

In a further aspect the invention provides a method of producing a plant with altered biomass,

the method comprising transformation of a plant with:
a) a polynucleotide encoding of a polypeptide with the amino acid sequence of SEQ ID NO:1 or a variant of the polypeptide, wherein the variant is capable of altering biomass in a plant;
b) a polynucleotide comprising a fragment, of at least 15 nucleotides in length, of the polynucleotide of a); or
c) a polynucleotide comprising a complement of the polynucleotide of a) or b).

In a further aspect the invention provides a method of producing a plant with altered seed yield,

the method comprising transformation of a plant with:
a) a polynucleotide encoding of a polypeptide with the amino acid sequence of SEQ ID NO:1 or a variant of the polypeptide, wherein the variant is capable of altering seed yield in a plant;
b) a polynucleotide comprising a fragment, of at least 15 nucleotides in length, of the polynucleotide of a); or
c) a polynucleotide comprising a complement of the polynucleotide of a) or b).

In a further aspect the invention provides a method of producing a plant with at least one of altered tolerance to at least one environmental stress selected from drought, cold, freezing heat and salinity,

the method comprising transformation of a plant with:
a) a polynucleotide encoding of a polypeptide with the amino acid sequence of SEQ ID NO:1 or a variant of the polypeptide, wherein the variant is capable of altering tolerance to at least one of the recited environmental stresses in a plant;
b) a polynucleotide comprising a fragment, of at least 15 nucleotides in length, of the polynucleotide of a); or
c) a polynucleotide comprising a complement of the polynucleotide of a) or b).

In one embodiment of each of the three aspects above, the isolated polynucleotide encodes a polypeptide with at least 70% identity to the sequence of SEQ ID NO:1.

In a further embodiment the polypeptide comprises an A20-type zinc finger domain and an AN1-type zinc finger domain.

Preferably the A20-type domain is in the N terminal half of the polypeptide.

Preferably the AN1-type domain is in the C terminal half of the polypeptide.

Preferably the A20-type domain has the general formula: X3-C-X(2-4)-C-X11-C-X2-C-X2, where X can be any amino acid.

Preferably the AN1-type domain has the general formula: C-X2-C-X(9-12)-C-X(1-2)-C-X4-C-X2-H-X5-H-X-C, where X can be any amino acid.

Preferably the A20-type domain has at least 70% identity to sequence of SEQ ID NO:16.

Preferably the A20-type domain comprises the sequence of SEQ ID NO:17.

Preferably the polypeptide comprises the sequence of SEQ ID NO:17.

Preferably the A20-type domain consists of the sequence of SEQ ID NO:16.

Preferably the AN1-type domain has at least 70% identity to sequence of SEQ ID NO:18.

Preferably the AN1-type domain comprises the sequence of SEQ ID NO:19.

Preferably the polypeptide comprises the sequence of SEQ ID NO:19.

Preferably the AN1-type domain consists of the sequence of SEQ ID NO:18.

Preferably the polypeptide is capable of modulating at least two, generally at least three, more preferably at least four, and most preferably all five of the recited environmental stresses.

In a further embodiment the polypeptide comprises the sequence of SEQ ID NO:1.

Preferably the plant is transformed with a genetic construct or vector comprising the polynucleotide.

In one embodiment the environmental stress is drought.

In a further embodiment the environmental stress is cold.

In a further embodiment the environmental stress is freezing.

In a further embodiment the environmental stress is heat.

In a further embodiment the environmental stress is salinity.

In a more preferred embodiment the polynucleotide of a) encodes a polypeptide with the amino acid sequence of SEQ ID NO: 1.

### Methods—Selection

In a further aspect the invention provides a method for selecting a plant with at least one of:
i) altered biomass,
ii) altered seed yield, and
iii) altered tolerance to at least one environmental stress selected from drought, cold freezing, heat and salinity,
   relative to suitable control plant, the method comprising testing of a plant for altered expression of a polynucleotide or polypeptide of the invention.

In a further aspect the invention provides a method for selecting a plant with altered biomass, relative to suitable control plant, the method comprising testing of a plant for altered expression of a polynucleotide or polypeptide of the invention.

In a further aspect the invention provides a method for selecting a plant with altered seed yield, relative to suitable control plant, the method comprising testing of a plant for altered expression of a polynucleotide or polypeptide of the invention.

In a further aspect the invention provides a method for selecting a plant with, increased tolerance to at least one environmental stress selected from drought, cold, freezing, heat and salinity, relative to suitable control plant, the method comprising testing of a plant for altered expression of a polynucleotide or polypeptide of the invention.

### Plants

In a further aspect the invention provides a plant cell or plant produced by the method of the invention.

In a further aspect the invention provides a group, or population, of plants selected by the method of the invention.

### Source of Polynucleotides of the Invention

The polynucleotides and polynucleotide variants of the invention may be derived from any species and/or may be produced synthetically or recombinantly.

In one embodiment the polynucleotide or variant, is derived from a plant species.

In a further embodiment the polynucleotide or variant, is derived from a gymnosperm plant species.

In a further embodiment the polynucleotide or variant, is derived from an angiosperm plant species.

In a further embodiment the polynucleotide or variant, is derived from a from dicotyledonous plant species.

In a further embodiment the polynucleotide or variant, is derived from a monocotyledonous plant species.

### Source of Plant Cells and Plants of the Invention

The plant cells and plants, of the invention may be derived from any species.

In one embodiment the plant cell or plant, is derived from a gymnosperm plant species.

In a further embodiment the plant cell or plant, is derived from an angiosperm plant species.

In a further embodiment the plant cell or plant, is derived from a from dicotyledonous plant species.

In a further embodiment the plant cell or plant, is derived from a monocotyledonous plant species.

Preferred dicotyledonous genera include: Amygdalus, Anacardium, Anemone, Arachis, Brassica, Cajanus, Cannabis, Carthamus, Carya, Ceiba, Cicer; Claytonia, Coriandrum, Coronilla, Corydalis, Crotalaria, Cyclamen, Dentaria, Dicentra, Dolichos, Eranthis, Glycine, Gossypium, Helianthus, Lathyrus, Lens, Lespedeza, Linum, Lotus, Lupinus, Macadamia, Medicago, Melilotus, Mucuna, Olea, Onobrychis, Ornithopus, Oxalis, Papaver, Phaseolus, Phoenix, Pistacia, Pisum, Prunus, Pueraria, Ribes, Ricinus, Sesamum, Thalictrum, Theobroma, Trifolium, Trigonella, Vicia and Vigna.

Preferred dicotyledonous species include: Amygdalus communis, Anacardium occidentale, Anemone americana, Anemone occidentalis, Arachis hypogaea, Arachis hypogea, Brassica napus Rape, Brassica nigra, Brassica campestris, Cajanus cajan, Cajanus indicus, Cannabis sativa, Carthamus tinctorius, Carya illinoinensis, Ceiba pentandra, Cicer arietinum, Claytonia exigua, Claytonia megarhiza, Coriandrum sativum, Coronilla varia, Corydalis flavula, Corydalis sempervirens, Crotalaria juncea, Cyclamen coum, Dentaria laciniata, Dicentra eximia, Dicentra formosa, Dolichos lablab, Eranthis hyemalis, Gossypium arboreum, Gossypium nanking, Gossypium barbadense, Gossypium herbaceum, Gossypium hirsutum, Glycine max, Glycine ussuriensis, Glycine gracilis, Helianthus annus, Lupinus angustifolius, Lupinus luteus, Lupinus mutabilis, Lespedeza sericea, Lespedeza striata, Lotus uliginosus, Lathyrus sativus, Lens culinaris, Lespedeza stipulacea, Linum usitatissimum, Lotus corniculatus, Lupinus albus, Medicago arborea, Medicago falcate, Medicago hispida, Medicago officinalis, Medicago sativa (alfalfa), Medicago tribuloides, Macadamia integrifolia, Medicago arabica, Melilotus albus, Mucuna pruriens, Olea europaea, Onobrychis viciifolia, Ornithopus sativus, Oxalis tuberosa, Phaseolus aureus, Prunus cerasifera, Prunus cerasus, Phaseolus coccineus, Prunus domestica, Phaseolus lunatus, Prunus. maheleb, Phaseolus mungo, Prunus. persica, Prunus. pseudocerasus, Phaseolus vulgaris, Papaver somniferum, Phaseolus acutifolius, Phoenix dactylifera, Pistacia vera, Pisum sativum, Prunus amygdalus, Prunus armeniaca, Pueraria thunbergiana, Ribes nigrum, Ribes rubrum, Ribes grossularia, Ricinus communis, Sesamum indicum, Thalictrum dioicum, Thalictrum flavum, Thalictrum thalictroides, Theobroma cacao, Trifolium augustifolium, Trifolium diffusum, Trifolium hybridum, Trifolium incarnatum, Trifolium ingrescens, Trifolium pratense, Trifolium repens, Trifolium resupinatum, Trifolium subterraneum, Trifolium alexandrinum, Trigonella foenumgraecum, Vicia angustifolia, Vicia atropurpurea, Vicia calcarata, Vicia dasycarpa, Vicia ervilia, Vaccinium oxycoccos, Vicia pannonica, Vigna sesquipedalis, Vigna sinensis, Vicia villosa, Vicia faba, Vicia sative and Vigna angularis.

Preferred monocotyledonous genera include: Agropyron, Allium, Alopecurus, Andropogon, Arrhenatherum, Asparagus, Avena, Bambusa, Bellavalia, Brimeura, Brodiaea, Bulbocodium, Bothrichloa, Bouteloua, Bromus, Calamovilfa, Camassia, Cenchrus, Chionodoxa, Chloris, Colchicum, Crocus, Cymbopogon, Cynodon, Cypripedium, Dactylis, Dichanthium, Digitaria, Elaeis, Eleusine, Eragrostis, Eremurus, Erythronium, Fagopyrum, Festuca, Fritillaria, Galanthus, Helianthus, Hordeum, Hyacinthus, Hyacinthoides, Ipheion, Iris, Leucojum, Liatris, Lolium, Lycoris, Miscanthis, Miscanthus×giganteus, Muscari, Ornithogalum, Oryza, Panicum, Paspalum, Pennisetum, Phalaris, Phleum, Poa, Puschkinia, Saccharum, Secale, Setaria, Sorghastrum, Sorghum, Thinopyrum, Triticum, Vanilla, X Triticosecale Triticale and Zea.

Preferred monocotyledonous species include: Agropyron cristatum, Agropyron desertorum, Agropyron elongatum, Agropyron intermedium, Agropyron smithii, Agropyron spicatum, Agropyron trachycaulum, Agropyron trichophorum, Allium ascalonicum, Allium cepa, Allium chinense, Allium porrum, Allium schoenoprasum, Allium fistulosum, Allium sativum, Alopecurus pratensis, Andropogon gerardi, Andropogon Gerardii, Andropogon scoparious, Arrhenatherum elatius, Asparagus officinalis, Avena nuda, Avena sativa, Bambusa vulgaris, Bellevalia trifoliate, Brimeura amethystina, Brodiaea californica, Brodiaea coronaria, Brodiaea elegans, Bulbocodium versicolor, Bothrichloa barbinodis, Bothrichloa ischaemum, Bothrichloa saccharoides, Bouteloua curipendula, Bouteloua eriopoda, Bouteloua gracilis, Bromus erectus, Bromus inermis, Bromus riparius, Calamovilfa longifilia, Camassia scilloides, Cenchrus ciliaris, Chionodoxa forbesii, Chloris gayana, Colchicum autumnale, Crocus sativus, Cymbopogon nardus, Cynodon dactylon, Cypripedium acaule, Dactylis glomerata, Dichanthium annulatum, Dichanthium aristatum, Dichanthium sericeum, Digitaria decumbens, Digitaria smutsii, Elaeis guineensis, Elaeis oleifera, Eleusine coracan, Elymus angustus, Elymus junceus, Eragrostis curvula, Eragrostis tef, Eremurus robustus, Erythronium elegans, Erythronium helenae, Fagopyrum esculentum, Fagopyrum tataricum, Festuca arundinacea, Festuca ovina, Festuca pratensis, Festuca rubra, Fritillaria cirrhosa, Galanthus nivalis, Helianthus annuus sunflower, Hordeum distichum, Hordeum vulgare, Hyacinthus orientalis, Hyacinthoides hispanica, Hyacinthoides non-scripta, Ipheion sessile, Iris collettii, Iris danfordiae, Iris reticulate, Leucojum aestivum, Liatris cylindracea, Liatris elegans, Lilium longiflorum, Lolium multiflorum, Lolium perenne, Lycoris radiata, Miscanthis sinensis, Miscanthus×giganteus, Muscari armeniacum, Muscari macrocarpum, Narcissus pseudonarcissus, Ornithogalum montanum, Oryza sativa, Panicum italiciunz, Panicum maximum, Panicum miliaceum, Panicum purpurascens, Panicum virgatum, Paspalum dilatatum, Paspalum notatum, Pennisetum clandestinum, Pennisetum glaucum, Pennisetum purpureum, Pennisetum spicatum, Phalaris arundinacea, Phleum bertolinii, Phleum pratense, Poa fendleriana, Poa pratensis, Poa nemoralis, Puschkinia scilloides, Saccharum officinarum, Saccharum robustum, Saccharum sinense, Saccharum spontaneum, Scilla autumnalis, Scilla peruviana, Secale cereale, Setaria italica, Setaria sphacelata, Sorghastrum nutans, Sorghum bicolor, Sorghum dochna, Sorghum halepense, Sorghum sudanense, Thinopyrum ponticum, Trillium grandiflorum, Triticum aestivum, Triticum dicoccum, Triticum durum, Triticum monococcum, Tulipa batalinii, Tulipa clusiana, Tulipa dasystemon, Tulipa gesneriana, Tulipa greigii, Tulipa kaufmanniana, Tulipa sylvestris, Tulipa turkestanica, Vanilla fragrans, X Triticosecale and Zea mays.

Other preferred plants are forage plant species from a group comprising but not limited to the following genera: Lolium, Festuca, Dactylis, Bromus, Thinopyrum, Trifolium, Medicago, Pheleum, Phalaris, Holcus, Lotus, Plantago and Cichorium.

Particularly preferred genera are Lolium or Trifolium. Particularly preferred are the species Lolium Perenne and Trifolium repens. Most preferred is the species Lolium perenne.

The term “plant” is intended to include a whole plant, any part of a plant, propagules and progeny of a plant.

The term ‘propagule’ means any part of a plant that may be used in reproduction or propagation, either sexual or asexual, including seeds and cuttings.

The plants of the invention may be grown and either selfed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the invention.

Preferably the plant, plant part, plant propagule or plant progeny contains the polynucleotide that was transformed into the parent plant. Preferably the plant, plant part, plant propagule or plant progeny expresses the polynucleotide that was transformed into the parent plant.

### DETAILED DESCRIPTION

### Definitions

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “environmental stress” includes at least one of the following stresses; drought, cold, freezing, heat and salinity.

The term “tolerance or tolerant to drought stress” is intended to describe a plant or plants which perform more favourably in any aspect of their growth and development under, or after, sub-optimal hydration conditions than do suitable control plants in the same conditions.

The term “tolerance or tolerant to cold stress” is intended to describe a plant or plants which perform more favourably in any aspect of their growth and development under, or after, sub-optimal-reduced reduced temperature conditions than do suitable control plants in the same conditions.

The term “tolerance or tolerant to freezing stress” is intended to describe a plant or plants that perform more favourably in any aspect of their growth and development under, or after, temperature conditions of less than or equal to 0° C., than do suitable control plants in the same conditions.

The term “tolerance or tolerant to heat stress” is intended to describe a plant or plants that perform more favourably in any aspect of their growth and development under, or after, sub-optimal elevated temperature conditions than do suitable control plants in the same conditions.

The term “tolerance or tolerant to salinity” is intended to describe a plant or plants that perform more favourably in any aspect of their growth and development under, or after, sub-optimal elevated salinity conditions than do suitable control plants in the same conditions.

With reference to the selection methods of the invention, a plant with increased tolerance to environmental stress refers to a plant, selected from a population of plants, which performs more favourably in any aspect of growth and development under stress conditions than does an average member of the population under the same conditions.

The more favourable performance referenced to above includes improved performance after the environmental stress is removed, that is improved recovery after a period of environmental stress.

The term “biomass” refers to the size and/or mass and/or number of vegetative organs of the plant at a particular age or developmental stage. Thus a plant with increased biomass has increased size and/or mass and/or number of vegetative organs than a suitable control plant of the same age or at an equivalent developmental stage. Conversely, a plant with decreased biomass has decreased size and/or mass and/or number of vegetative organs than a suitable control. Altered biomass may also involve an alteration in rate of growth and/or rate of formation of vegetative organs during some or all periods of the life cycle of a plant relative to a suitable control. Thus altered biomass may result in an advance or delay in the time taken for such a plant to reach a certain developmental stage.

The term “seed yield” refers to the size and/or mass and/or number of seed or grain produced by a plant. Thus a plant with increased seed yield has increased size and/or mass and/or number of seed or grain relative to a suitable control plant at the same age or an equivalent developmental stage. Conversely, a plant with decreased seed yield has increased size and/or mass and/or number of seed or grain relative to a suitable control plant the same age or an equivalent development stage.

The term “altered” with reference to seed yield is intended to encompass either a decrease or increase in seed yield.

The term “modulating” with reference to seed yield is intended to encompass either decreasing or increasing seed yield.

Suitable control plants may include non-transformed plants of the same species or variety, or plants of the same species or variety transformed with a control construct.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides, more preferably at least 60 nucleotides, more preferably at least 70 nucleotides, more preferably at least 80 nucleotides, more preferably at least 90 nucleotides, more preferably at least 100 nucleotides, more preferably at least 150 nucleotides, more preferably at least 200 nucleotides, more preferably at least 250 nucleotides, more preferably at least 300 nucleotides, more preferably at least 350 nucleotides, more preferably at least 400 nucleotides, more preferably at least 450 nucleotides of contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods of the invention.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence, that is complementary to the probe, in a hybridization-based assay. The probe may consist of a “fragment” of a polynucleotide as defined herein.

### Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 20 nucleotide positions, preferably at least 50 nucleotide positions, more preferably at least 100 nucleotide positions, and most preferably over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.5 [Nov 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:
bl2seq-i nucleotideseq1-j nucleotideseq2-F F-p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-2′77) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [Nov 2002]) from NCBI (ftp://ftp.ncbi.nih.gov/blast/).

The similarity of polynucleotide sequences may be examined using the following unix command line parameters:

bl2seq-i nucleotideseq1-j nucleotideseq2-F F-p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰ , more preferably less than 1×10⁻¹⁰⁰, more preferably less than 1×10⁻¹¹⁰, and most preferably less than 1×10⁻¹²⁰ when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for a polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec 6; 254(5037):1497-500) Tm values are higher than those for DNA-DNA or DNA-RNA hybrids, and can be calculated using the formula described in Giesen et al., Nucleic Acids Res. 1998 Nov 1; 26(21):5004-6. Exemplary stringent hybridization conditions for a DNA-PNA hybrid having a length less than 100 bases are 5 to 10° C. below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [Nov 2002]) from NCBI (ftp://ftp.ncbi.nih.gov/blast/) via the tblastx algorithm as previously described.

The function of a variant polynucleotide of the invention in modulating biomass and/or tolerance to environmental stress plant may be assessed by methods known to those skilled in the art and described in the Examples section. Function may be assessed for example by altering expression of the polynucleotide in a plant by methods known in the art and/or described herein, and, analyzing performance of the transformed plant in comparison to a control plant, under or after conditions of environmental stress; and in non-stressed conditions for assessment of altered biomass. Further plant transformation protocols for several species are known to those skilled in the art. A list of such protocols is provided herein.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [Nov 2002]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention.

Polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [Nov 2002]) from NCBI (ftp://ftp.ncbi.nih.gov/blast/). The similarity of polypeptide sequences may be examined using the following unix command line parameters:

bl2seq-i peptideseq1-j peptideseq2 -F F-p blastp

Variant polypeptide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰, more preferably less than 1×10⁻¹⁰⁰, more preferably less than 1×10⁻¹¹⁰, more preferably less than 1×10⁻¹²⁰ and most preferably less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

A “transgene” is a polynucleotide that is taken from one organism and introduced into a different organism by transformation. The transgene may be derived from the same species or from a different species as the species of the organism into which the transgene is introduced.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 by spacer between the repeated regions.

A “transgenic plant” refers to a plant which contains new genetic material as a result of genetic manipulation or transformation. The new genetic material may be derived from a plant of the same species as the resulting transgenic plant or from a different species.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

The applicants have identified a polynucleotide from ryegrass (SEQ ID NO:7) which encodes a polypeptide (SEQ ID NO:1) which modulates biomass and/or tolerance to at least one environmental stress selected from drought, cold, freezing, heat and salinity in plants. The applicants have also identified polynucleotide variants of SEQ ID NO:7 (SEQ ID NO:8-12) which encode polypeptide variants of SEQ ID NO:1 (SEQ ID NO:2-6) which modulate biomass and tolerance to at least one environmental stress selected from drought, cold, freezing, heat and salinity in plants.

The applicants have identified the presence both of an A20-type and as AN1-type zinc finger motif in the polypeptide of SEQ ID NO:1, and each of the polypeptide variants. The applicants have also identified sequence motifs that are completely conserved in each of the zinc finger motifs, in all of the polypeptide sequences and variants.

The invention provides plants altered, relative to suitable control plants, in biomass and tolerance to at least one environmental stress selected from drought, cold, freezing, heat and salinity. The invention provides both plants with both increased biomass and stress tolerance and plants with decreased biomass and stress tolerance. The invention also provides methods for the production or selection of such plants.

### Methods for Isolating or Producing Polynucleotides

The polynucleotide molecules of the invention can be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides, of the invention or useful in the methods of the invention, include use of all, or portions of, the polynucleotides set forth herein as hybridization probes. The technique of hybridizing labeled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion and oligonucleotide synthesis.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species. Variants (including orthologues) may be identified by the methods described.

### Methods for Identifying Variants

### Physical Methods

Variant polynucleotides may be identified using PCR-based methods (Mullis et al., Eds. 1994 The Polymerase Chain Reaction, Birkhauser). Typically, the polynucleotide sequence of a primer, useful to amplify variant polynucleotide molecules PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor

Press, 1987) or by identifying polypeptides from natural sources with the aid of such antibodies.

### Computer Based Methods

Polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [Nov 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the invention, including variant polypeptides, may be prepared using peptide synthesis methods well known in the art such as direct peptide synthesis using solid phase techniques (e.g. Stewart et al., 1969, in Solid-Phase Peptide Synthesis, W H Freeman Co, San Francisco Calif., or automated synthesis, for example using an Applied Biosystems 431A Peptide Synthesizer (Foster City, Calif.). Mutated forms of the polypeptides may also be produced during such syntheses.

The polypeptides and variant polypeptides of the invention may also be purified from natural sources using a variety of techniques that are well known in the art (e.g. Deutscher, 1990, Ed, Methods in Enzymology, Vol. 182, Guide to Protein Purification).

Alternatively the polypeptides and variant polypeptides of the invention may be expressed recombinantly in suitable host cells and separated from the cells as discussed below.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Constructs and Vectors

The invention provides a host cell which comprises a genetic construct or vector of the invention. Host cells may be derived from, for example, bacterial, fungal, insect, mammalian or plant organisms.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

Host cells of the invention may also be useful in methods for production of an enzymatic product generated by an expressed polypeptide of the invention. Such methods may involve culturing the host cells of the invention in a medium suitable for expression of a recombinant polypeptide of the invention, optionally in the presence of additional enzymatic substrate for the expressed polypeptide of the invention. The enzymatic product produced may then be separated from the host cells or medium by a variety of art standard methods.

### Methods for Producing Plant Cells and Plants Comprising Constructs and Vectors

The invention further provides plant cells which comprise a genetic construct of the invention, and plant cells modified to alter expression of a polynucleotide or polypeptide of the invention. Plants comprising such cells also form an aspect of the invention.

Production of plants altered in biomass and/or tolerance to environmental stress may be achieved through methods of the invention. Such methods may involve the transformation of plant cells and plants, with a construct designed to alter expression of a polynucleotide or polypeptide capable of modulating biomass and/or tolerance to environmental stress in such plant cells and plants. Such methods also include the transformation of plant cells and plants with a combination of constructs designed to alter expression of one or more polypeptides or polypeptides capable of modulating biomass and/or tolerance to environmental stress in such plant cells and plants.

Methods for transforming plant cells, plants and portions thereof with polynucleotides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of strategies for genetically manipulating plants are available (e.g. Birch, 1997, Ann Rev Plant Phys Plant Mol Biol, 48, 297). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Exemplary terminators that are commonly used in plant transformation genetic construct include, e.g., the cauliflower mosaic virus (CaMV) 35S terminator, the Agrobacterium tuinefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenbert. Eds) Springer Verlag. Berline, pp. 325-336.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide of the invention may include an antisense copy of a polynucleotide of the invention. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

| | |
|---|---|
| 5′GATCTA 3′ | 3′CTAGAT 5′ |
| (coding strand) | (antisense strand) |
| 3′CUAGAU 5′ | 5′GAUCUCG 3′ |
| mRNA | antisense RNA |

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 bp between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

The term genetic construct as used herein also includes small antisense RNAs and other such polynucleotides useful for effecting gene silencing.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a polynucleotide of the invention is also contemplated.

The polynucleotide inserts in genetic constructs designed for gene silencing may correspond to coding sequence and/or non-coding sequence, such as promoter and/or intron and/or 5′ or 3′ UTR sequence, or the corresponding gene.

Other gene silencing strategies include dominant negative approaches and the use of ribozyme constructs (McIntyre, 1996, Transgenic Res, 5, 257)

Pre-transcriptional silencing may be brought about through mutation of the gene itself or its regulatory elements. Such mutations may include point mutations, frameshifts, insertions, deletions and substitutions.

The following are representative publications disclosing genetic transformation protocols that can be used to genetically transform the following plant species: Rice (Alam et al., 1999, Plant Cell Rep. 18, 572); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, : 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5, 63,174 and 5,750,871); cereals (U.S. Pat. No. 6,074,877); and ryegrass (Bajaj et al., 2006, Plant Cell Reports, 25: 651-659). Other species are contemplated and suitable methods and protocols are available to in the scientific literature for use by those skilled in the art.

Several further methods known in the art may be employed to alter expression of a nucleotide and/or polypeptide of the invention. Such methods include but are not limited to Tilling (Till et al., 2003, Methods Mol Biol, 2%, 205), so called “Deletagene” technology (Li et al., 2001, Plant Journal 27(3), 235) and the use of artificial transcription factors such as synthetic zinc finger transcription factors. (e.g. Jouvenot et al., 2003, Gene Therapy 10, 513). Additionally antibodies or fragments thereof, targeted to a particular polypeptide may also be expressed in plants to modulate the activity of that polypeptide (Jobling et al., 2003, Nat. Biotechnol., 21(1), 35). Transposon tagging approaches may also be applied. Additionally peptides interacting with a polypeptide of the invention may be identified through technologies such as phase-display (Dyax Corporation). Such interacting peptides may be expressed in or applied to a plant to affect activity of a polypeptide of the invention. Use of each of the above approaches in alteration of expression of a nucleotide and/or polypeptide of the invention is specifically contemplated.

### Methods for Selecting Plants

Methods are also provided for selecting plants with altered biomass and/or tolerance to environmental stress. Such methods involve testing of plants for altered for the expression of a polynucleotide or polypeptide of the invention. Such methods may be applied at a young age or early developmental stage when the altered biomass and/or tolerance to environmental stress may not necessarily be visible, to accelerate breeding programs directed toward improving biomass and/or tolerance to environmental stress.

The expression of a polynucleotide, such as a messenger RNA, is often used as an indicator of expression of a corresponding polypeptide. Exemplary methods for measuring the expression of a polynucleotide include but are not limited to Northern analysis, RT-PCR and dot-blot analysis (Sambrook et al., Molecular Cloning: A Laboratory Manual, 2nd Ed. Cold Spring Harbor Press, 1987). Polynucleotides or portions of the polynucleotides of the invention are thus useful as probes or primers, as herein defined, in methods for the identification of plants with altered tolerance to environmental stress. The polypeptides of the invention may be used as probes in hybridization experiments, or as primers in PCR based experiments, designed to identify such plants.

Alternatively antibodies may be raised against polypeptides of the invention. Methods for raising and using antibodies are standard in the art (see for example: Antibodies, A Laboratory Manual, Harlow A Lane, Eds, Cold Spring Harbour Laboratory, 1998). Such antibodies may be used in methods to detect altered expression of polypeptides which modulate tolerance to environmental stress in plants. Such methods may include ELISA (Kemeny, 1991, A Practical Guide to ELISA, NY Pergamon Press) and Western analysis (Towbin & Gordon, 1994, J Immunol Methods, 72, 313).

These approaches for analysis of polynucleotide or polypeptide expression and the selection of plants with altered expression are useful in conventional breeding programs designed to produce varieties with altered biomass and/or tolerance to environmental stress.

### Plants

The plants of the invention may be grown and either self-ed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

FIG. 1 shows the map of a vector CORF136, for plant transformation, comprising ORF136 operably linked to a constitutive double CaMV35S promoter (D35S P).

FIG. 2 shows the map of a vector DORF136, for plant transformation, comprising ORF136 operably linked to a ryegrass dehydrin-like promoter.

FIG. 3 shows the sequence of the CORF vector presented in FIG. 1. The ORF136 coding sequence is shown in bold. The double CaMV35S promoter is shown in italics. UTR (untranslated region) sequence is shown with underlining.

FIG. 4 shows the sequence of the DORF136 vector presented in FIG. 2. The ORF136 coding sequence is shown in bold. The dehydrin-like promoter is shown in italics. UTR (untranslated region) sequence is shown with underlining.

FIG. 5 shows alignment of the ORF136 polypeptide and variants thereof. Completely conserved residues are highlighted by asterisks. Also shown is the position of the A20-type zinc finger motif ITLCANRCGFPGNPATQNLCQNCFL (SEQ ID NO: 16) in ORF136. Also shown is the position of the AN1-type zinc finger motif CSSCWKRVGLTGFRCRCGELFCGAHRYSDRHGC (SEQ ID NO: 18) in ORF136. Also highlighted is a motif (CGFPGNPAT—SEQ ID NO: 17) within the A20-type motif that is completely conserved in all of the sequences. Also highlighted is a motif (RVGLTGFRCRC—SEQ ID NO: 19) within the AN1-type motif that is completely conserved in all of the sequences.

FIG. 6 shows a phylogram of the protein sequences aligned in FIG. 5.

FIG. 7 shows the condition of the transgenic and non-transgenic plants prior to the application of drought-stress.

FIG. 8 shows the condition of non-stressed plants (top left background) and the stressed plants (foreground) at the end of 10-days of drought-stress and 4 days of recovery.

FIG. 9 shows a graph depicting altered biomass in the transgenic plants over the non-transgenic control during recovery after drought. Plant lines 7ae1 to 7ae17, are also described as DORF136-1 to DORF136-17, respectively; and GUS line is also described as line expressing D35S::GUS(bacterial uidA gene), which served as a “non-gene” transgenic control.

FIG. 10 shows a graph depicting altered biomass in the transgenic plants over the non-transgenic control during fully watered conditions. Plant lines 7ael to 7ae17, are also described as DORF136-1 to DORF136-17, respectively.

FIG. 11 shows southern-blot analysis for gene integration number determination.

FIG. 12 shows a graphical representation of the increase in seed yield in T₁ transformed plants due to altered expression of the AN 1 and A20 containing Zinc Finger TF ORF136 (=ORF138).

### EXAMPLES

The invention will now be illustrated with reference to the following non-limiting examples.

### Example 1

### Identification of Polynucleotides Which Modulate Biomass Production, and Tolerance to Environmental Stresses

Introduction:

Perennial ryegrass (Lolium perenne L.) is a cool temperate pasture plant from the family Gramineae and the tribe Festucaceae. To generate a profile of relative gene expression patterns in ryegrass, RNA was extracted from samples obtained from ambient temperature growth, cold grown, hydrated, dehydrated and rehydrated or dehydration pre- and post-grazed plants during autumn, summer, spring and winter, and used for constructing a SAGE (serial analysis of gene expression) (Velculescu et al. 1995, Science 270: 484-487) library.

### Materials and Methods

Perennial ryegrass (Lolium perenne L.) cv. Bronsyn was used throughout this study. Field grown samples were collected from active paddocks at Dexcel, Hamilton, New Zealand during the peak of each season. Grass samples were collected from pre-grazed (15 days post grazing) and post-grazed (1 day post grazing) ryegrass swards. Tufts of grass samples were harvested from 3-6 randomly chosen sites and stored in dry-ice after snap-freezing with liquid nitrogen. During spring, immature spike and floral initials were also harvested. For stress-treatment, the following conditions were used on lab-grown ryegrass: Mature lab-grown perennial ryegrass that was grown in growth chamber for 15 months at 85% RH, 20° C./18° C. and 16 h/8 h day/night regime; Hydrated control grown for 55 days at 85% RH, 20° C./18° C. and 16 h/8 h day/night regime; 6 days at 70% RH, 22° C./16° C. and 16 h/8 h day/night regime, seedlings were kept watered throughout their life; Dehydrated sample watered only for 55 days at 85% RH, 20° C./18° C. and 16 h/8 h day/night regime; 3 days at 70% RH, 28° C./20° C. and 16 h/8 h day/night regime; 3 days at 50% RH, 28° C./20° C. and 16 h/8 h day/night regime; Rehydrated samples were from dehydrated plants that was watered for 24 hours and grown at 70% RH, 22° C./16° C. and 16 h/8 h day/night regime; Cold-stressed plants were grown for 55 days at 85% RH, 20° C./18° C. and 16 h/8 h day/night regime; 7 days at 70% RH, 22° C./16° C. and 16 h/8 h day/night regime; 7 days at 70% RH, 6° C./2° C. and 16 h/8 h day/night regime, seedlings were kept watered throughout their life.

### Construction of SAGE Libraries

RNA was extracted using TRIZOL® reagent (Invitrogen, Calif., USA) and by the protocol described by the manufacturer from tissue that was ground in liquid nitrogen. For each SAGE library 100 μg of total RNA was used and the libraries were created using I-SAGE™ or I-SAGE™ Long kit (Invitrogen, Calif., USA) according to manufacturer's protocol. From each library 960-1,920 clones were sequenced (Australian Genome

Research Facility, Brisbane, Australia) and the tags extracted using the SAGE2000 software.

### SAGE Bioinformatics:

The relational database was designed to hold tags, libraries and expression counts of the SAGE experiments. Each tag sequence (including enzyme sequence) was searched against the whole Ryegrass non-overlapping Gene thresher and the EST sets. The search was carried out in both direction and used exact match only. Results were loaded to the relational database using General Feature Format (GFF) approach (http://www3.ebi.ac.uk/Services/WebFeat).

All Ryegrass Gene thresher and the EST sequences were annotated using homology searches against some or all the following public and propriety databases:
AGI TIGR Gene Indices, Arabidopsis, release 11, Jan. 2004
OGI TIGR Gene Indices, Rice, release 14-1, Jan. 2004
GENESEQN Derwent patent DNA sequences 2002, Dec. 7
GENESEQP Derwent patent amino acid sequences 2002, Dec 7
Os_unigene Oryza sativa Unigene unique sequences 2004, Mar. 18
est_others Other EST sequences (mammal, fungi, prokaryote) 2003, Mar. 11
est_plant Viridiplantae subset of Non-redundant Database of GenBank+EMBL+DDBJ EST Divisions 2004, Mar. 15
nr All non-redundant GenBank CDS translations+PDB+SwissProt+PIR 2003, Mar. 11
nr_plant Plant subset of HS subset of BT subset of All non-redundant GenBank CDS translations+PDB+SwissProt+PIR 2003, Aug. 8
nt All Non-redundant GenBank+EMBL+DDBJ+PDB sequences (but no EST, STS, GSS, or HTGS sequences) 2003, Mar. 11
nt_monocots Monocot subset of All Non-redundant GenBank+EMBL+DDBJ+PDB sequences (but no EST, STS, GSS, or HTGS sequences) 2003, Mar. 11
swissprot The last major release of the SWISS-PROT protein sequence database (no updates) 2003, Mar. 28

A cutoff of E value less than E-05 was used and maximum of 10 targets per database were stored in the relational database.

### Tags Annotation:

Tags with hits to the Ryegrass sets were annotated by creating a summary of all the annotations of the involved sequences. The summary was generated using an algorithm to calculate the frequency of the occurrence of each word in the annotations and rank them in descending order based on the number off occurrences. The summary was limited to 10 words and a void word list was used to filter out insignificant information. The resulting summary line was used as an indication of what the tags were likely to be. Actual numbers are displayed; giving additional information that could be used to evaluate the significance of each of the words in the summary. This method of automatic annotation using keyword counts is similar to the Automatic comment that is used by the ProDom database (available on the world wide web at http://protein.toulouse.inra.fr/prodom/current/html/home.php) to annotate the automatically generated protein domain families.

Detailed annotation based on the top hits of the involved sequences was displayed when viewing tags data.

A polynucleotide sequence of particular interest was identified by carrying out a BLASTX analysis of the polynucleotide sequences, which had SAGE tags exclusive to the dehydrated perennial ryegrass SAGE library, against the putative transcription factors. The analysis resulted in the identification of a Zinc-finger like protein gene, ORF136. A cDNA for ORF136 is shown in SEQ ID NO:7. The ORF136 coding sequences extends from nucleotide position 88 to nucleotide position 576. The transcript profile in our SAGE library is

Data for SAGE Tag CCTGCGGCAG (SEQ ID NO: 13)

| SAGE_TAG | CCTGCGGCAG | tpm* |
|---|---|---|
| Winter Pre-grazed | 0 | 0 |
| Winter Post-grazed | 0 | 0 |
| Winter roots | 0 | 0 |
| Spring Pre-grazed | 0 | 0 |
| Spring Post-grazed | 0 | 0 |
| Inflorescence | 0 | 0 |
| Summer Post-grazed | 0 | 0 |
| Autumn Pre grazed | 0 | 0 |
| Autumn Post-grazed | 0 | 0 |
| Mature | 0 | 0 |
| Cold-stressed | 0 | 0 |
| Hydrated | 0 | 0 |
| Dehydrated | 1 | 59 |
| Rehydrated | 0 | 0 |

*tpm = Tag counts per million tags

The following primers were used to amplify ORF136 (based on CLP0023004352-cF3_{—}20040205)

| 827_ORF136_f | |
|---|---|
| AAGCCAGCCAGACTCTCTCTCGTACC | (SEQ ID NO: 14) |
| 828_ORF136_r | |
| GCACCTTCAGTTTCCTCCGTTCATTC | (SEQ ID NO: 15) |

### Example 2

### Identification Variants of ORF136

The ORF136 polynucleotide sequence was used as seed sequence to perform a discontiguous megablast BLASTN search against i) GenBank nucleotide collection NR/NT database (v2.2.17 release date Aug. 26, 2007) and ii) patent sequences database (v2.2.17 release date Aug. 26, 2007).

The polypeptide sequence encoded by the ORF136 was used as seed sequence to perform Position-Specific Iterated BLASTP search against GenBank NR database (v2.2.17 release date Aug. 26, 2007). A USPTO search was also performed against UniRef100 protein database at EBI (v2.2.15 release date Oct. 15, 2006). This gene appears to encode a protein similar in low homology to rice stress-associated protein gene as described by (Mukhopadhyay et al., 2004, PNAS (USA) 101(16):6309-6314).

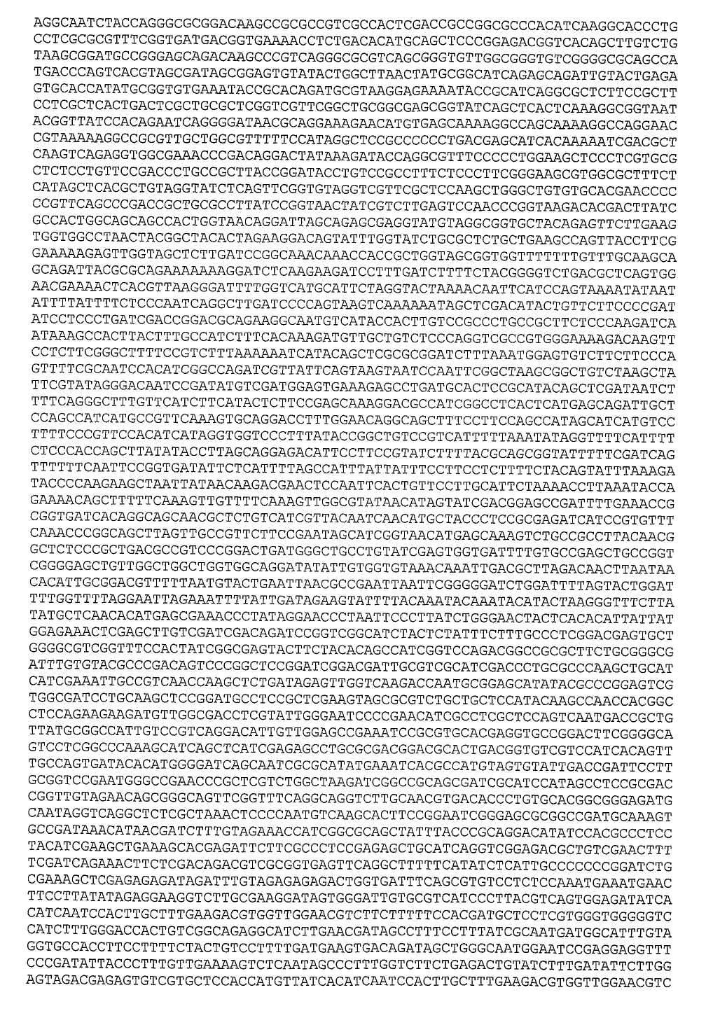

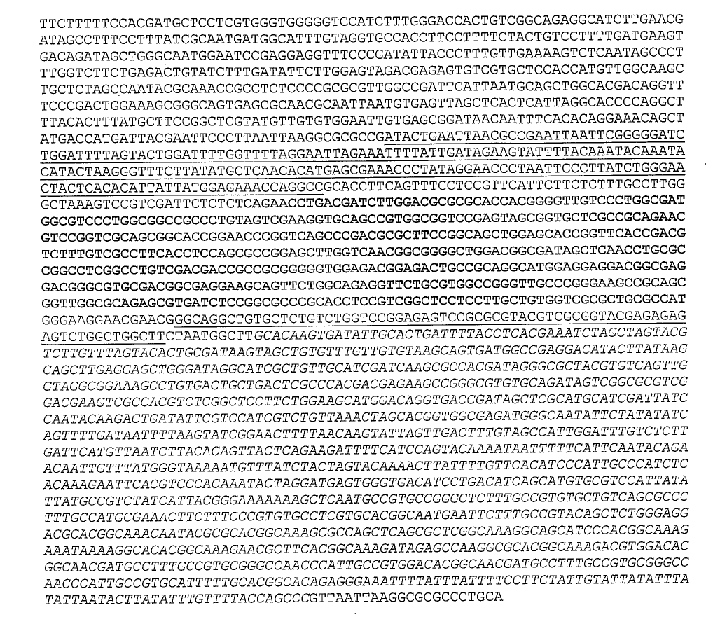

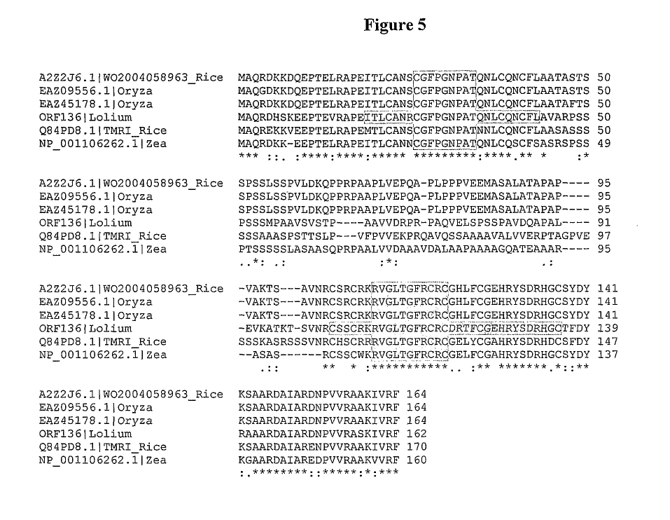

Variant sequences were aligned using the EMBOSS tool EMMA (Thompson, J. D., Higgins, D. G. and Gibson, T. J. 1994, CABIOS, 10, 19-29.), which is an interface to the popular multiple alignment program ClustalW. Aligned sequences were visualised using another EMBOSS tool called prettyplot, which displays aligned sequences with colouring and consensus sequences marked in a separate line. The alignment is shown in FIG. 5.

ORF136 and the variant proteins aligned in FIG. 5 appear to be zinc-finger transcription factors and are characterised by the presence of an A20-type zinc finger (pfam01754=X3-C-X(2-4)-C-X11-C-X2-C-X2, where X can be any amino acid; Marchler-Bauer A, et al, 2007, Nucleic Acids Res. 35: D237-40) motif in the N terminal half of the protein, and an AN1-type zinc finger motif (c101438=C-X2-C-X(9-12)-C-X(1-2)-C-X4-C-X2-H-X5 -H-X-C Where X can be any amino acid; Marchler-Bauer A, et al, 2007, Nucleic Acids Res. 35: D237-40) in the C terminal half of the protein.

The extent of the A20-type motif in the ORF136 protein is highlighted in FIG. 5. The sequence of the A20 motif from ORF136 is shown in SEQ ID NO:16. This motif is well conserved in all the variants with 20 of the 25 residues being completely conserved. The applicants also identified a motif within the A20-type that is completely conserved in all of the aligned sequences. The position of this completely conserved motif is shown in FIG. 5, and the sequence is shown in SEQ ID NO:17.

The extent of the AN1-type motif in the ORF136 protein is highlighted in FIG. 5. The sequence of the AN1-type motif from ORF136 is shown in SEQ ID NO:18. This motif is well conserved in all the variants with 23 of the 33 residues being completely conserved. The applicants also identified a motif within the AN1-type that is completely conserved in all of the aligned sequences. The position of this completely conserved motif is shown in FIG. 5, and the sequence is shown in SEQ ID NO:19.

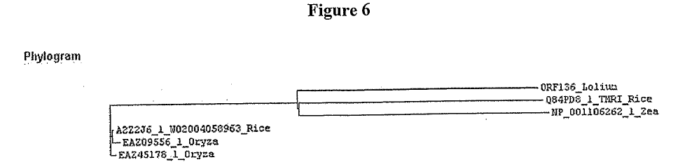

A phylogram of the protein sequences aligned in FIG. 5 is shown in FIG. 6.

The phylogram was produced by aligning the SEQ ID NOs 1-6 using the default parameters set in the ClustalW sequence analysis tool at the European Bioinformatics Institute website (http://www.ebi.ac.uk/Tools/clustalw2/).

### Example 3

### Preparation of Vectors Comprising Polynucleotides of the Invention for Plant Transformation, and Transformation of Plants

Vectors comprising ORF136 (CORF136 and DORF136)

Vectors for over-expressing ORF136 were produced by standard molecular biology techniques. A vector in which ORF136 is driven by a double 35S promoter was designated CORF136. A vector in which a ryegrass dehydrin like promoter drives ORF136 was designated DORF136.

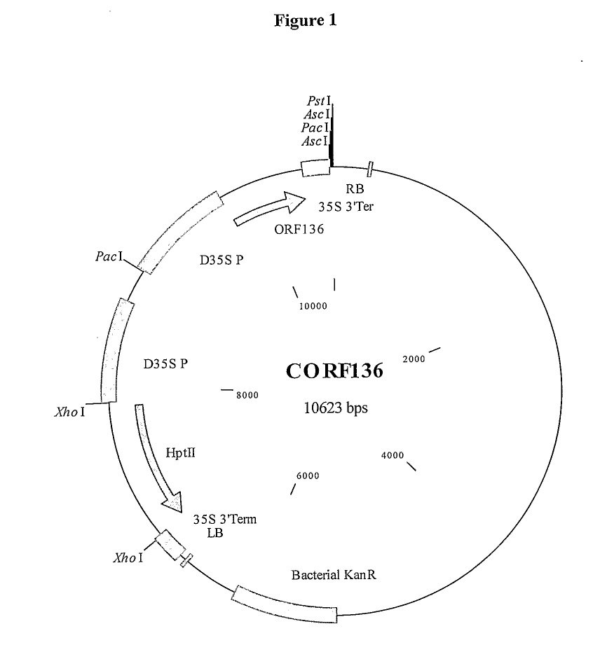

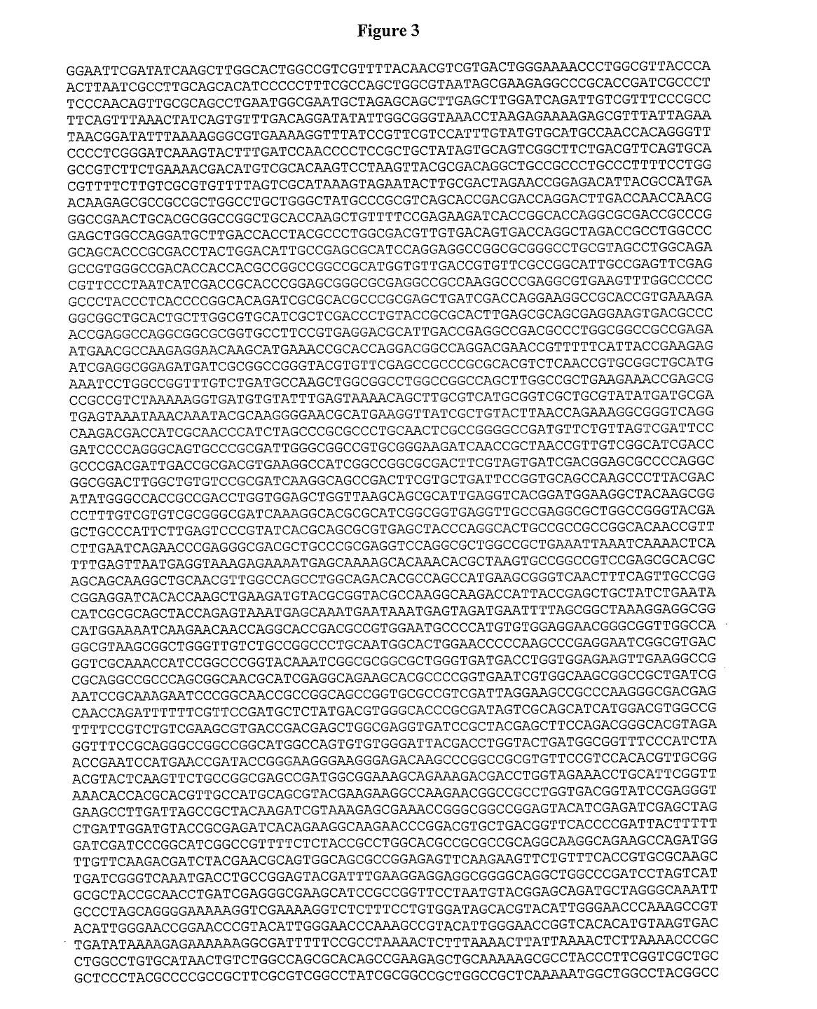

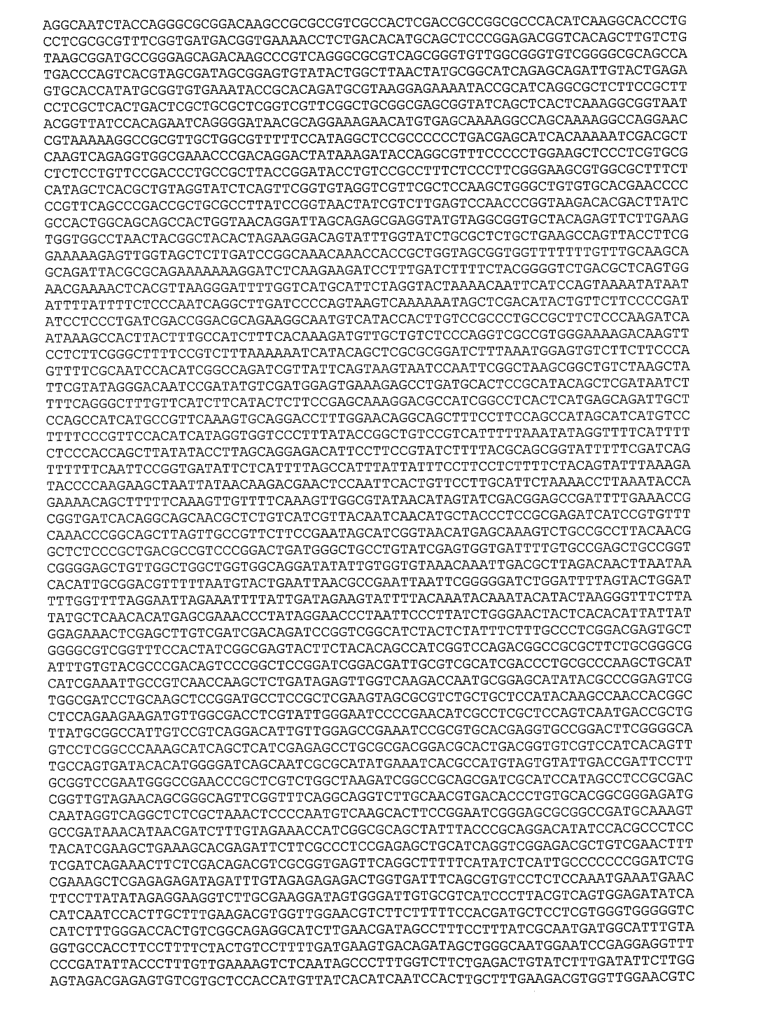

A map of (CORF136) is shown in FIG. 1. The sequence of CORF136 is shown in FIG. 3, and in SEQ ID NO:20.

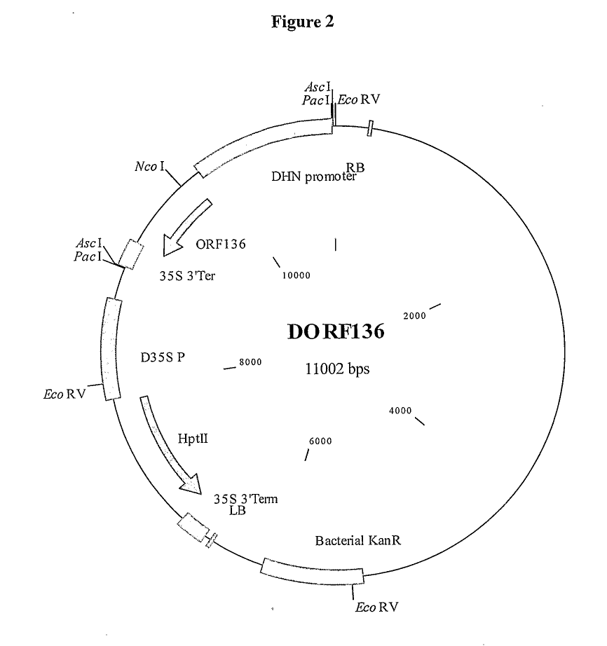

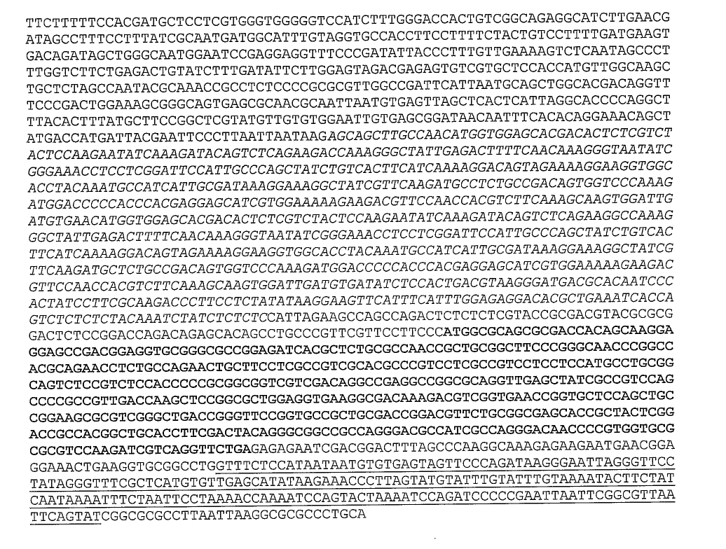

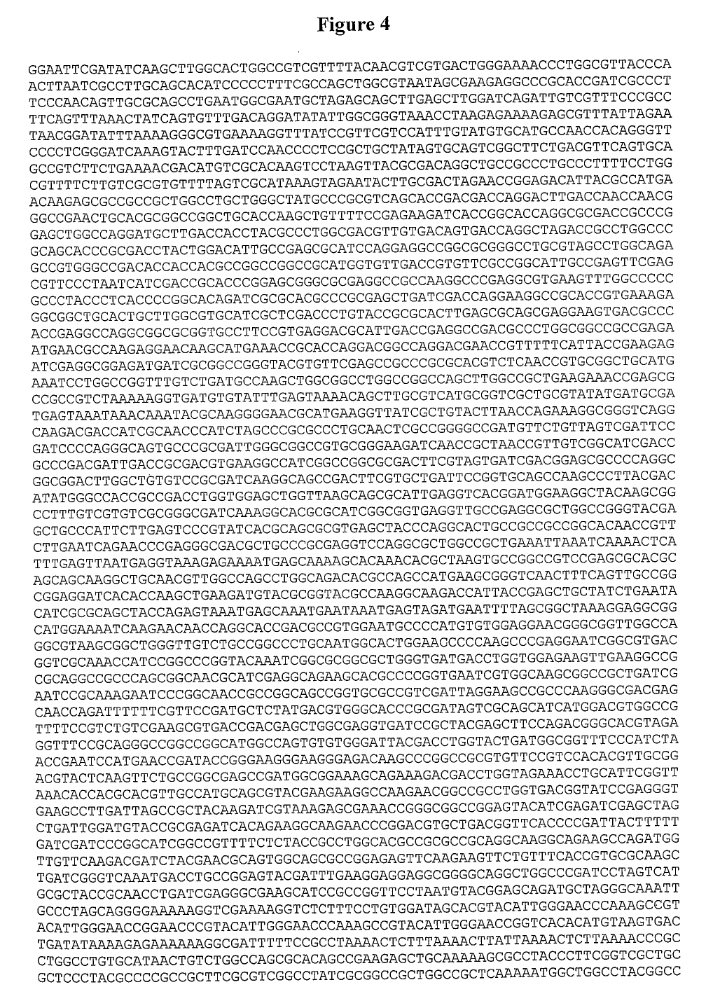

A map of DORF136 is shown in FIG. 2. The sequence of DORF136 is shown in FIG. 4, and SEQ ID NO:21.

### Transformation of Plants With the Polynucleotides of the Invention

Perennial ryegrass (Lolium perenne L. cv. Impact) was transformed essentially as described in Bajaj et. al. (Plant Cell Reports, 2006, 25: 651-659). Embryogenic callus derived from mersitematic regions of the tillers of selected ryegrass lines and Agrobacterium tumefaciens strain EHA101 carrying a modified binary vector (CORF136, FIG. 1 or DORF136, FIG. 2) was used for transformation experiments. Embryogenic calli were immersed with overnight-grown Agrobacterium cultures for 30 minutes with continuous shaking. Calli resistant to hygromycin were selected after subculturing them on co-cultivation medium for 4 weeks. After selection, the resistant calli were subcultured on regeneration medium every 2 weeks until the plants regenerated. The regenerants that continued to grow after two or three rounds of selection proved to be stable transformants. Each regenerated plant was then multiplied on maintenance medium to produce clonal plantlets and subsequently rooted on MS medium without hormones. A rooted plant from each clone was transferred into contained glasshouse conditions while retaining a clonal counterpart in tissue culture as backup. Seventeen independent transgenic lines (7ae1 to 7ae17, also described as DORF136-1 to DORF136-17, respectively) have been analyzed in a climate-controlled environmental laboratory, where they were assessed for biomass under fully watered condition. The separate clones of these lines were also subjected to drought stress.

### Hybridisation by Southern Blot

Genomic DNA was isolated from transformed and non-transformed control lines of Lolium perenne. Approximately 1.5 g of leaf blade and pseudostem material was harvested from each line. Tissue was ground to a powder in a mortar and pestle with liquid Nitrogen and stored in a 50 mL tube at −80° C. until extraction. DNA was isolated from the prepared tissue essentially as described in Doyle and Doyle, 1990 (Doyle J. J. and Doyle J. L. 1990. Isolation of plant DNA from fresh tissue. Focus 12:13-15). Extracted DNA was resuspended in 800 μl TE and the concentration was estimated using a Nanodrop N1000.

Approximately 25 μg genomic DNA's were digested with restriction enzymes EcoRV and SpeI. DNA was digested overnight at 37° C. with 40 units of restriction enzyme in a 100 μl reaction volume. A further 20 units of enzyme was added after 12 hours incubation and digested for another 2 hours. The digest reaction was then precipitated with Ethanol, centrifuged, the supernatant discarded, air dried and resuspended in 25 μl dH₂O for electrophoresis.

Digested DNA samples were electrophoretically separated for approximately 4 hours at 45 volts on a 10×15 cm agarose gel using a 1×TAE running buffer. Following electrophoresis the gel was denatured (1.5 M NaCl, 0.5 M NaOH) then neutralised (1.5 MNaCl, 0.5 M Tris-base) before capillary transfer to positively charged nylon membrane (Hybond N⁺) using the alkali method as described by the supplier (GE Healthcare, Buckinghamshire, England). The transferred DNA's were fixed to the membrane using Stratalinker (Stratagene, La Jolla, Calif., USA) following the manufacturer's recommendations. Membranes were stored at 4° C. between blotting paper in a plastic bag until required.

A DNA probe was synthesised using the PCR-based labelling reaction incorporating alkali-labile digoxigenin-11-dUTP (DIG, Roche Diagnostics, Basel, Switzerland). Template DNA was amplified from the hygromycin phosphotransferase gene using primers rghlcpf (5′-3′, AATACGAGGTCGCCAACATCT, SEQ ID NO:22) and rghcpr (5′-3′, AGGAACCCTAATTCCCTTATCTG, SEQ ID NO:23) as described by Bajaj et al 2006 (Plant Cell Reports).

Nylon membranes were pre-hybridised using the DIG Easy Hyb Kit (Roche Diagnostics, Basel, Switzerland) for 1 hour at 42° C. The DIG-labelled probe was denatured (95□C, 5 minutes) and added to the pre-hybridisation solution and the membranes hybridised for approximately 12 hours at 42° C.

Following hybridisation membranes were subjected to two 5 minute washes in Low Stringency Wash Buffers (2×SSC, 0.1% SDS (w/v)) at room temperature with shaking, followed by two 15 minute washes in High Stringency Wash Buffer (0.1×SSC, 0.1% SDS (w/v)). Further washes were performed using the DIG and Wash and Block Buffer Set (Roche Diagnostics, Basel, Switzerland). Membranes were then incubated for 30 minutes at room temperature with Blocking Solution containing anti-DIG-AP antibody (Roche Diagnostics, Basel, Switzerland). Hybridised probes were then detected with the application of CDP-Star (Roche Diagnostics, Basel, Switzerland) chemiluminescent substrate for alkaline phosphatase. Membranes were heat-sealed in a plastic bag, exposed to X-ray film (Kodak BioMax MS, Rochester, N.Y.) ranging from 60 minutes to overnight and in a 100 Plus automatic developer.

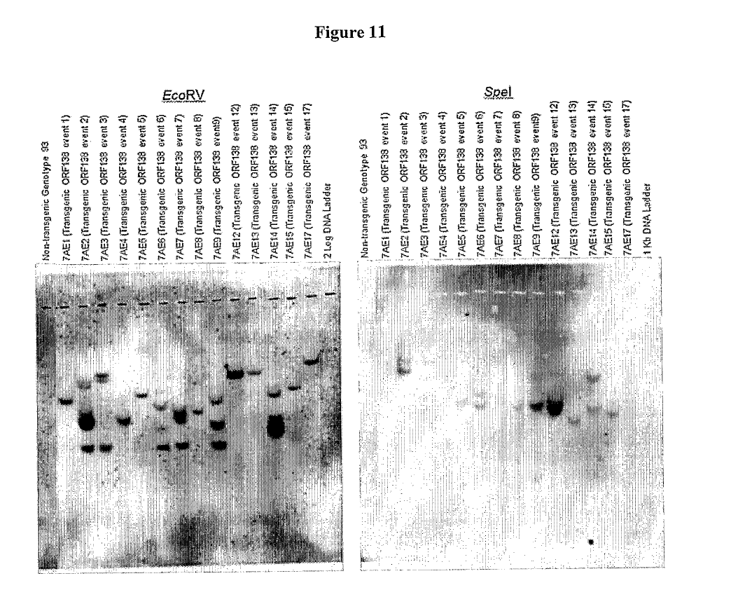

The results in FIG. 11, show that each of the ORF136 transgenic events (labeled as TAE1-9, TAE12-15 and TAE17 [note that ORF138=ORF136 here] are transgenic for the P DORF136 T-DNA.

### Example 4

### Alteration of Tolerance to Environmental Stress in Plants Transformed With Polynucleotides of the Invention

### Drought Screening in Growth Chamber

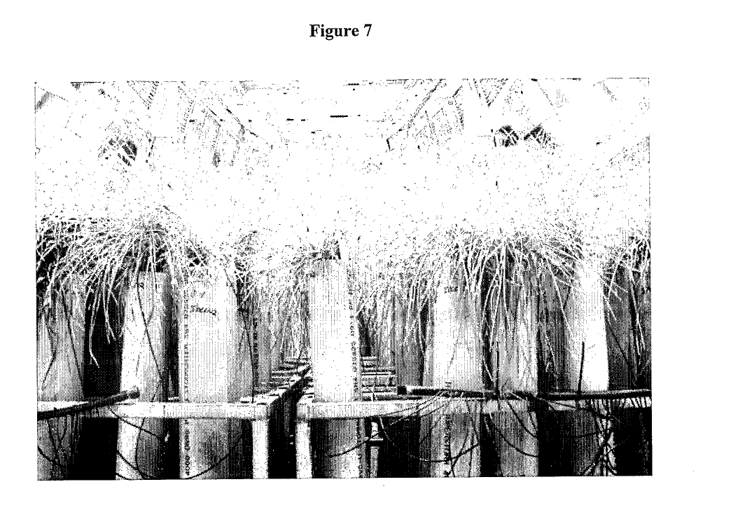

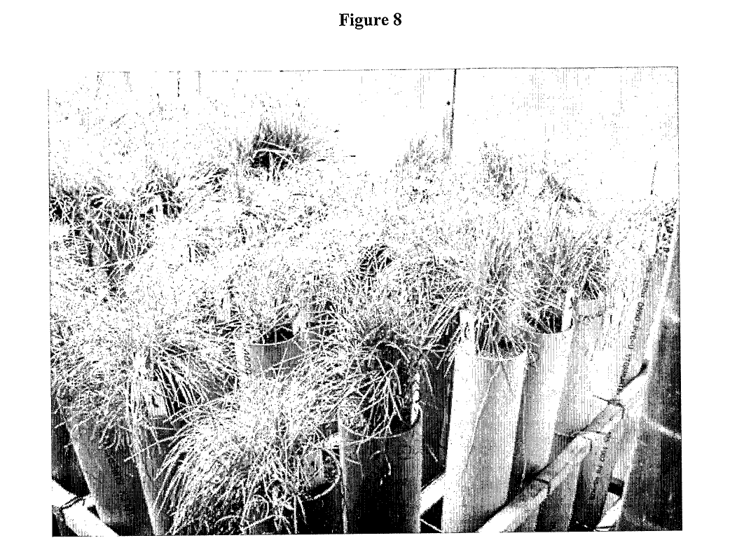

A plant growth system was built of one meter long; 90 mm diameter plastic storm-water pipes. The pipes were placed on a mobile tray and supported at the sides by ropes and metal frame. The tubes were plugged at the bottom with rockwool and progressively filled with washed mortar sand using water to achieve uniform packing. At the center of the open end of each tube a clump of perennial ryegrass (25 tillers) was planted. There were three independent transgenic events and each event was planted in six tubes. The plants were arranged at random, one in each of the six replicates, and grown at 70% relative humidity; 16/8 hours day/night cycle and under 650 μmol.m⁻².s⁻¹ light intensity. The plants were irrigated daily once in the morning with 50 mL Hoagland's solution (Hoagland and Arnon, 1938) and again in the afternoon with 50 mL plain water. The plants were acclimated initially for fourteen days (FIG. 7) and then the plants were trimmed back to 15 cm height. All plants were allowed to recover from trimming for the next seven days. Drought-stress was imposed only on three of the six replicates after this recovery period by withholding the application of Hoagland's solution and water. During the drought screening, all plants were subjected to 50% relative humidity; 16/8 hours day/night cycle and 650 μmol.m⁻².s⁻¹ light intensity. The drought-stress was carried out for eight days in the first round when the volumetric water content in the sandy soil was less than 1% at 12 cm depth. The volumetric water content in the control, hydrated plants was greater than 10% at 12 cm depth. The drought-stress was stopped and irrigation resumed to the drought-stressed plants. All plants were also returned to 70% relative humidity; 16/8 hours day/night cycle and 650 μmol.m⁻².s⁻¹ light intensity. After four days (FIG. 8), plant height was measured and then trimmed down to 15 cm height. The fresh-weight of the trimmed sample was measured and the samples were dried down. The plants were allowed to grow under frilly watered conditions for total of 14 days and again subject to drought stress for 14 days. This cycle was conducted three times and in the third cycle, the drought period was for 21 days.

### Soil Moisture Monitoring

The soil moisture (VWC, volumetric water content) was recorded with a Field Scout™ TDR 100 Soil Moisture Meter (Spectrum Technologies, Inc., IL, USA) as necessary. Measurements were taken in each tube at 12 cm depth and the average of three readings recorded. Following the establishment period subsurface irrigation was cut off. Soil moisture content declined and reached volumetric water content (VWC) below 1.0%. A period of no-irrigation was followed by a re-growth period.

### Above-Ground Biomass

Leaf clipping dry weight was determined before (>10% VWC) and after drought stress (<1.0% VWC). All leaves were cut at 15 cm clipping height. The fresh weights (FW) of leaves were measured immediately, then leaves were dried at 60° C. for 96 h and the dry weight (DW) was measured. The ability to grow under drought-stress is calculated as percentage of inverse mass loss, which is calculated as the difference of fresh weight in non-stressed and drought-stressed conditions over fresh weight in non-stressed condition, i.e. (1-[{Fresh weight (or dry weight) in non-stressed condition—Fresh weight (or dry weight) in drought-stressed condition}/Fresh weight (or dry weight) in non-stressed condition]) %.

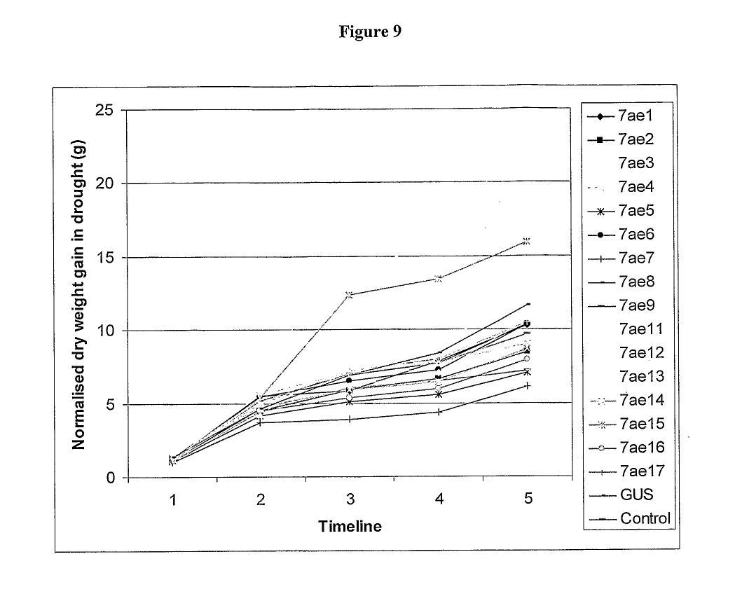

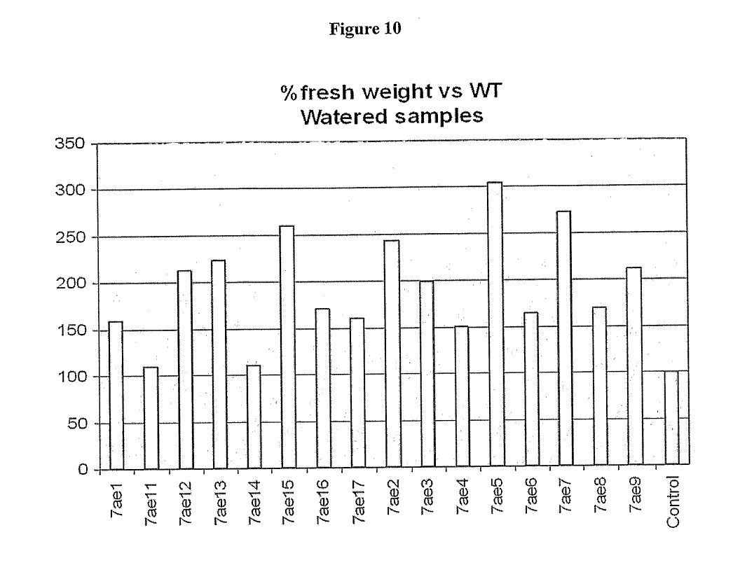

Transgenic plants grew more vigorously than the control plant both in hydrated and drought-stressed conditions (FIGS. 9 and 10). They also recovered better than the non-transgenic control. Increase in plant biomass in the transgenic line DORF136-12 (7ae12) and 15 (7ae15) were significantly higher than the control during drought and recovery, i.e the entire experimental duration (FIG. 9). These plants also performed better than the control when analyzed for growth in non-stress conditions (FIG. 10).

### Example 5

### Alteration of Biomass of Plants Transformed With Polynucleotides of the Invention

The procedure described in Example 4 was also carried out for the transgenic and wild type plants under fully hydrated conditions. Several of the transgenic lines produced more biomass than wild type plants in each of the harvest (essentially as described in Example 4). When cumulative growth was determined over a period of four harvests, Lines DORF136-5, 12 and 15 consistently produced more biomass than corresponding control (non-transgenic lines), Line DORF136-5 (7ae5), 7 (7ae7) and 15 (7ae15), for example showed a 250-300% increase in biomass over the non-transgenic lines (FIG. 10).

### Example 6

### Alteration of Seed Yield of Plants Transformed With Polynucleotides of the Invention

### Transgenic Seed Production Under Controlled Conditions

Prior to vernalization, plants were maintained in tissue culture by regular micropropagation. A single ramet of each independently transformed line was transferred to PC2 Containment greenhouse. Once established plants were grown in PB3/4 polybags in a general purpose potting medium (60:40 peat:sand). Thrive foliar fertiliser (Yates New Zealand, Auckland) was applied at a weekly interval. Plants were trimmed on a regular basis to a height of approximately 40 mm to prevent flowering.

Genotypes of the cultivar Impact (Accession No. A10745, Margot Forde Germplasm

Centre, Palmerston North, New Zealand) were grown as crossing partners of in preparation for controlled crosses to primary (T₀) transgenics. Vernalisation was initiated when plants —transgenic lines and crossing partners—had developed a minimum of 20 tillers. Plants were moved from the greenhouse to a chilled chamber set at 6° C. degrees and 8 hour photoperiod for a period of 12 weeks. Lighting was provided by 400 w SonT Agro high pressure sodium lamps (Philips Lighting, Mairangi Bay, Auckland, New Zealand).

Following vernalisation florally induced plants were returned to the greenhouse (20-25° C., 16 hour photoperiod) for development of floral tillers. Inflorescence stems emerged from tillers after 3-4 weeks of growth. Up to eight inflorescence stems at an evenly matched developmental stage, from the primary transgenic and crossing partner, were combined in a polyester crossing bag prior to the emergence of anthers or stigma from florets. Crossing bags were retained on plants for approximately three months until senescence of inflorescence stems was observed. At this stage the inflorescences of each crossing partner was harvested separately into paper bags and incubated in an oven set at 28° C. for two weeks.

Seeds were separated from the florets of harvested material by rubbing in a seed mill between two corrugated rubber surfaces. Large chaff was removed from the seed by passage through a seed sieve. Finally the seed was cleaned in a South Dakota Seed Blower (Seedburo Equipment Co., Chicago, USA). Seed collected from individual crosses was counted and weighed.

Segregation of the T-DNA in seedlings of the T₁ generation was determined by PCR using primers designed to amplify a fragment of the hygromycin phosphotransferase gene. Production of T₂ generation seed was performed using the protocol as described above by controlled crosses of seedling transgenic T₁ progeny and Impact (A10745) genotypes.

See from T₀ and T₁ lines were collected, weighted and cultured for each line.

TABLE 1
Seed yield in T₀ plant from single copy insertion transgenic lines

| Female parent | Pollon donor | Seeds collected |
|---|---|---|
| 7AE1 | A10745 | 0 |
| A10745 | 7AE1 | 2 |
| 7AE4 | A10745 | 119 |
| A10745 | 7AE4 | 280 |
| 7AE5 | A10745 | 64 |
| A10745 | 7AE5 | 136 |
| 7AE5 | A10745 | 84 |
| A10745 | 7AE5 | 146 |
| 7AE7 | A10745 | 1 |
| A10745 | 7AE7 | 0 |
| 7AE8 | A10745 | 13 |
| A10745 | 7AE8 | 21 |
| 7AE13 | A10745 | 15 |
| A10745 | 7AE13 | 0 |
| 7AE15 | A10745 | 72 |
| A10745 | 7AE15 | 358 |
| 7AE17 | A10745 | 20 |
| A10745 | 7AE17 | 451 |

Prior to the emergence of anthers or stigma from florets, the inflorescence stems from the primary transgenic line and seed-derived non-transgenic crossing partner were combined, at an evenly matched developmental stage, in a polyester crossing bag. Crossing bags were retained on plants until senescence of inflorescence stems were observed. At this stage the inflorescences of each crossing partner was harvested separately and seeds harvested after additional drying in an incubator.

TABLE 2
Seed yield in T₁ generation from single copy insertion transgenic lines

| | Average number of | Average seed |
|---|---|---|
| T₁ Line | Seeds/per plant | Yield (g)/plant |
| 7AE4 | 363 | 0.6182 |
| 7AE5 | 205 | 0.3496 |
| 7AE8 | 137 | 0.233 |
| 7AE13 | 144 | 0.2443 |
| 7AE15 | 211 | 0.3588 |
| 7AE17 | 25 | 0.0425 |
| ORF138 Average | 181 | 0.3077 |
| Transgenic Control 1 | 61 | 0.1036 |
| Transgenic Control 2 | 119 | 0.2035 |
| Transgenic Control Average | 90 | 0.154 |

Prior to the emergence of anthers or stigma from florets, the inflorescence stems from each transgenic progeny plant were combined in a polyester crossing bag at an evenly matched developmental stage with the inflorescence stems of different seed-derived non-transgenic crossing partner. Crossing bags were retained on plants until senescence of inflorescence stems was observed. At this stage the inflorescences stems of both crossing partners were harvested together and seeds harvested after additional drying in an incubator. With the sole exception of the transgenic event 7AE8, which produced only one transgenic progeny plant, the seed yield for each transgenic event was calculated from averaging the seed yields from more than three transgenic progeny plants and their respective crossing partners. The seed yield data is compared against the average seed yield from two different transgenic-even progeny plant-crosses stemming from the transformation of ryegrass plants with a different gene.

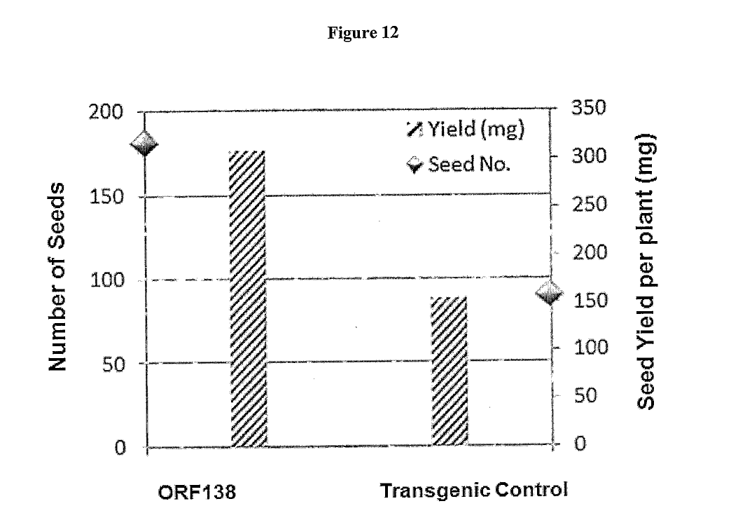

A graphical representation of the increased seed yield in T₁ plants transformed with ORF136 (=ORF138), taken from the average data in Table 2 above, is shown in FIG. 12.

The above examples illustrate practice of the invention. It will be appreciated by those skilled in the art that numerous variations and modifications may be made without departing from the spirit and scope of the invention.

SUMMARY OF SEQUENCES:

| SEQ | | | |
|---|---|---|---|
| ID | Sequence | | |
| NO: | type | Information | Species |
| 1 | polypeptide | ORF136 | Lolium perenne |
| 2 | polypeptide | gb/A2Z2J6.1 | Oryza sativa |
| | | | Indica |
| 3 | polypeptide | gb/EAZ09556.1 | Oryza sativa |
| | | | Indica |
| 4 | polypeptide | EAZ45178.1 | Oryza sativa |
| 5 | polypeptide | Q84PD8.1\|TMRI | Oryza sativa |
| 6 | polypeptide | NP_001106262.1 | Zea mays |
| 7 | polynucleotide | ORF136 cDNA | Lolium perenne |
| 8 | polynucleotide | gb/AF140722/A2Z2J6.1 | Oryza sativa |
| | | | Indica |
| 9 | polynucleotide | gb/CM000134/EAZ09556.1 | Oryza sativa |
| | | | Indica |
| 10 | polynucleotide | gb/CM000146/EAZ45178.1 | Oryza sativa |
| 11 | polynucleotide | gb/AY601878/Q84PD8.1\|TMRI | Oryza sativa |
| 12 | polynucleotide | NM_001112791/ | Zea mays |
| | | NP_001106262.1 | |
| 13 | polynucleotide | SAGE tag | Lolium perenne |
| 14 | polynucleotide | primer | Lolium perenne |
| 15 | polynucleotide | primer | Lolium perenne |
| 16 | polynucleotide | A20 motif | Lolium perenne |
| 17 | polynucleotide | A20 conserved motif | Artificial |
| 18 | polynucleotide | AN1 motif | Lolium perenne |
| 19 | polynucleotide | AN1 conserved motif | Artificial |
| 20 | polynucleotide | CORF136 vector | Artificial |
| 21 | polynucleotide | DORF136 vector | Artificial |
| 22 | polynucleotide | rghlcpf primer | Artificial |
| 23 | polynucleotide | rghcpr primer | Artificial |

1-43. (canceled)

44. An isolated polynucleotide encoding a polypeptide comprising the sequence of SEQ ID NO: 1 or a variant thereof comprising a sequence with at least 82% identity to SEQ ID NO: 1, wherein % identity is calculated over the whole length of SEQ ID NO: 1.

45. The isolated polynucleotide of claim 44 wherein the variant is a polypeptide capable of modulating in a plant at least one of:
i) biomass,
ii) seed yield, and
iii) tolerance to at least one environmental stress selected from drought, cold, freezing, heat and salinity.

46. The isolated polynucleotide of claim 44, wherein the polypeptide or variant comprises an A20-type zinc finger domain and an AN1-type zinc finger domain.

47. The isolated polynucleotide of claim 46, wherein the A20-type domain is in the N terminal half of the polypeptide.

48. The isolated polynucleotide of claim 46, wherein the AN1-type domain is in the C terminal half of the polypeptide.

49. The isolated polynucleotide of claim 46, wherein the A20-type domain has the general formula: X3-C-X(2-4)-C-X11-C-X2-C-X2, where X can be any amino acid.

50. The isolated polynucleotide of claim 46, wherein the AN1-type domain has the general formula: C-X2-C-X(9-12)-C-X(1-2)-C-X4-C-X2-H-X5-H-X-C, where X can be any amino acid.

51. The isolated polynucleotide of claim 46, wherein the A20-type domain has at least 70% identity to sequence of SEQ ID NO:16.

52. The isolated polynucleotide of claim 46, wherein the A20-type domain comprises the sequence of SEQ ID NO:17.

53. The isolated polynucleotide of claim 46, wherein the A20-type domain comprises the sequence of SEQ ID NO:16.

54. The isolated polynucleotide of claim 46, wherein the AN1-type domain has at least 70% identity to sequence of SEQ ID NO:18.

55. The isolated polynucleotide of claim 46, wherein the AN1-type domain comprises the sequence of SEQ ID NO:19.

56. The isolated polynucleotide of claim 46, wherein the AN1-type domain comprises of the sequence of SEQ ID NO:18.

57. The isolated polynucleotide of claim 44, wherein the isolated polynucleotide encodes a polypeptide with the sequence of SEQ ID NO:1.

58. The isolated polynucleotide of claim 44, wherein the isolated polynucleotide comprises a sequence with at least 70% identity to the coding sequence of SEQ ID NO:7.

59. The isolated polynucleotide of claim 44, wherein the isolated polynucleotide comprises a sequence capable of hybridising under stringent conditions to the coding sequence of SEQ ID NO:7.

60. The isolated polynucleotide of claim 44, wherein the isolated polynucleotide comprises the coding sequence of SEQ ID NO: 7.

61. The isolated polynucleotide of claim 44 comprising the sequence of SEQ ID NO: 7 or a variant thereof comprising a sequence with at least 82% identity to the coding sequence of SEQ ID NO: 7, wherein the variant encodes a polypeptide capable of modulating in a plant at least one of:
i) biomass,
ii) seed yield, and
iii) tolerance to at least one environmental stress selected from drought, cold, freezing, heat and salinity.

62. The isolated polynucleotide of claim 61, wherein the isolated polynucleotide comprises the coding sequence of SEQ ID NO: 7.

63. A polypeptide encoded by a polynucleotide of claim 44.

64. An isolated polynucleotide comprising a fragment, of at least 200 nucleotides in length, of a polynucleotide of claim 44.

65. A genetic construct, or expression construct, comprising a polynucleotide of claim 44.

66. A host cell comprising a genetic construct or expression construct of claim 65.

67. A host cell genetically modified to express a polynucleotide of claim 44.

68. A plant cell comprising a genetic construct or expression construct of claim 65.

69. A plant which comprises a plant cell of claim 68.

70. A method of producing a plant with at least one of:
i) altered biomass,
ii) altered seed yield, and
iii) altered tolerance to at least one environmental stress selected from drought, cold, freezing, heat and salinity,
the method comprising transformation of a plant cell or plant with:
a) a polynucleotide of claim 44;
b) a polynucleotide comprising a fragment, of at least 200 nucleotides in length, of the polynucleotide of a); or
c) a polynucleotide comprising a complement of the polynucleotide of a) or b).

71. The method of claim 70 where both biomass and tolerance to at least one of the recited environmental stresses is altered in the plant.

72. The method of claim 70 wherein said alteration is an increase.

73. The method of claim 70, wherein the polynucleotide in iii) a) encodes a polypeptide comprising the sequence of any one of SEQ ID NO: 2 to 4 and 6.

74. A method for selecting a plant with at least one of:
i) altered biomass,
ii) altered seed yield, and
iii) altered tolerance to at least one environmental stress selected from drought, cold freezing, heat and salinity,
relative to suitable control plant, the method comprising testing of a plant for altered expression of a polynucleotide of claim 44 or a polypeptide encoded by the polynucleotide.

75. A plant cell or plant, produced by the method of claim 70, that is genetically modified to comprise a polynucleotide encoding a polypeptide comprising the sequence of SEQ ID NO: 1 or a variant thereof comprising a sequence with at least 82% identity to SEQ ID NO: 1, wherein % identity is calculated over the whole length of SEQ ID NO: 1.

76. A group, or population, of plants selected by the method of claim 74.

77. A part, fruit, seed, harvested material, propagule or progeny of a plant of claim 69.

78. A part, fruit, seed, harvested material, propagule or progeny of a plant of claim 69, that is genetically modified to comprise at least one polynucleotide encoding a polypeptide comprising the sequence of SEQ ID NO: 1 or a variant thereof comprising a sequence with at least 82% identity to SEQ ID NO: 1, wherein % identity is calculated over the whole length of SEQ ID NO: 1.
